(19) United States
(12) Patent Application Publication
(10) Pub. No.: US 2025/0339546 A1
(43) Pub. Date: Nov. 6, 2025

(54) GLIOBLASTOMA TUMOR GROWTH INHIBITON BY SAT1 KNOCKDOWN

(71) Applicants: BIOMARK CANCER SYSTEMS, INC., Richmond (CA); THE UNIVERSITY OF MANITOBA, Winnipeg (CA)

(72) Inventors: Donald MILLER, Winnipeg (CA); Vinith YATHINDRANATH, Winnipeg (CA); Rashid BUX, Richmond (CA)

(21) Appl. No.: 18/701,085

(22) PCT Filed: Oct. 14, 2022
(86) PCT No.: PCT/CA2022/051521

§ 371 Date: Apr. 12, 2024

Related U.S. Application Data
(60) Provisional application No. 63/262,523, filed on Oct. 14, 2021.

(51) Int. Cl. A61K 47/64; A61K 47/60; C12N 15/113

(52) U.S. Cl. CPC A61K 47/6425; A61K 47/60; C12N 15/1137; C12Y 203/01057; C12N 2310/14

(57) ABSTRACT

Described herein is a biocompatible lipid nanoparticle (LNP) composition suitable for delivering RNA payloads into cells and tissues of a subject. The biocompatible LNPs comprise an ionizable cationic lipid as a core component and have a net neutral surface charge at physiological pH. Delivery of LNP-encapsulated siRNA inhibiting the expression Spermidine/spermine N1-acetyltransferase 1 (SAT1) is shown to inhibit proliferation of a glioblastoma cell line, but not in other cells pertinent to brain tissue such as microvascular endothelial cells, primary human astrocytes, and macrophage cells. Use of a cadherin-binding peptide to increase delivery of LNP-encapsulated siRNA across a blood-brain barrier monolayer model is also described.

The present description relates to biocompatible lipid nanoparticles (LNP) based on ionizable cationic lipids for encapsulating and delivering RNA payloads into cells and tissues of a subject, as well as the use of cadherin binding peptides to enhance LNP delivery across the blood-brain barrier. The present description also relates to the use of LNP-encapsulated siRNA to knockdown expression of Spermidine/spermine N1-acetyltransferase 1 (SAT1), leading to preferential reduced proliferation in glioblastoma cells.

The present description refers to a number of documents, the contents of which are herein incorporated by reference in their entirety.

### BACKGROUND

Glioblastoma multiforme (GBM) is the most common type of primary brain tumor in adults. As a grade IV astrocytoma, GBM is a highly invasive and aggressive form of tumor. With current treatments, including surgical resection of the tumor, radiation and chemotherapy, the median survival time of patients is about 15 months. Despite the advances in cancer medicine, the prognosis of GBM patients has not seen a notable improvement over the last two decades. The development of novel treatments for GBM remains difficult due to several complicating factors, including the fact that GBM tumor cells are often resistant to conventional therapies, the brain is susceptible to damage from such conventional therapies and has a limited capacity to repair itself, and that many drugs cannot cross the blood-brain barrier to act on the GBM tumor. Thus, there is a great need for novel therapies that can cross the blood-brain barrier, inhibit GBM tumor growth, but at the same time exert minimal adverse effects on non-GBM brain cells.

### SUMMARY

In a first aspect, described herein is a biocompatible lipid nanoparticle composition comprising, or consisting essentially of, an siRNA encapsulated in a lipid component, the lipid component comprising a mixture of: (a) an ionizable cationic lipid (e.g., ionizable cationic unsaturated lipid) having a polar head group with a pKa of below 7; (b) a PEGylated lipid; (c) a sterol; and (d) a phospholipid.

In a further aspect, described herein is a method for inhibiting the growth of brain tumor cells, the method comprising contacting the brain tumor cells with a biocompatible lipid nanoparticle composition comprising an siRNA for inhibiting expression of Spermidine/spermine N1-acetyltransferase 1 (SAT1), encapsulated in a lipid component.

In a further aspect, described herein is a cadherin binding peptide for use in improving the delivery of RNA (e.g., siRNA) encapsulated in a biocompatible lipid nanoparticle composition.

### General Definitions

Headings, and other identifiers, e.g., (a), (b), (i), (ii), etc., are presented merely for ease of reading the specification and claims. The use of headings or other identifiers in the specification or claims does not necessarily require the steps or elements be performed in alphabetical or numerical order or the order in which they are presented.

The use of the word “a” or “an” when used in conjunction with the term “comprising” in the claims and/or the specification may mean “one” but it is also consistent with the meaning of “one or more”, “at least one”, and “one or more than one”.

The term “about”, when used herein, indicates that a value includes the standard deviation of error for the device or method being employed in order to determine the value. In general, the terminology “about” is meant to designate a possible variation of up to 10%. Therefore, a variation of 1, 2, 3, 4, 5, 6, 7, 8, 9 and 10% of a value is included in the term “about”. Unless indicated otherwise, use of the term “about” before a range applies to both ends of the range.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, advantages and features of the present description will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 shows a Kaplan curve of the overall survival of GBM patients with high and low SAT1 expression.

FIG. 2 shows a Kaplan curve of the progress-free survival of GBM patients with high and low SAT1 expression.

FIGS. 3A and 3B show schematic illustrations of the formulation of lipid nanoparticle (LNP)-siRNA following micro-mixing in a microfluidic chamber. The flow rate ratio (FRR) of aq:org of 1:3 and a total flow rate of 12 mL/min yielded siRNA encapsulated LNPs that were in the 80 nm (polydispersity index [PDI]=0.16) size range with neutral surface charge.

FIG. 4 shows the result on SAT1 expression of LNP-siSAT1 transfection in U251 glioblastoma cells in comparison to control U251 cells that were transfected with a negative control LNP-siSCR (scrambled SAT1 sequence), after 72 hours. FIG. 4A shows the result of SAT1 mRNA expression (n=3, p<0.05). FIG. 4B shows the result of SAT1 protein expression (n=3, p<0.05).

FIG. 5 shows the effect of SAT1 knockdown on the number of viable U251 glioblastoma cells 72 hours after transfection with “Control” (LNP-siSCR) or “LNP-siSAT1” (SAT1-inhibiting) LNP-siRNA formulations. (n=3, p<0.05).

FIGS. 6A and 6B show the effect of SAT1 knockdown on the number of viable of glioblastoma cells (U251), brain microvascular endothelial cells (hCMEC/D3), primary human astrocytes (HA), and macrophage cells (ANA-1) (FIG. 6A) and SAT1 mRNA expression levels (FIG. 6B) 72 hours after transfection with “Control” (LNP-siSCR) or “LNP-siSAT1” (SAT1-inhibiting) LNP-siRNA formulations (n=3).

FIGS. 7A and 7B show the effect on viability/proliferation of a cytotoxic response to chemotherapy and radiation in various cells following exposure to LNP with scrambled siRNA (“Control”) or SAT1 siRNA (“LNP_siSAT1”) in U251 cells (GBM) (FIG. 7A), hCMEC/D3 (brain endothelial cells) (FIG. 7B), ANA-1 (macrophage) (FIG. 7C), and primary human astrocyte cells (HA) (FIG. 7D) exposed to chemotherapeutic agents carmustine (100 μM), Doxorubicin (i.e., DOX) (0.1 μM) and Topotecan (i.e., TOPO) (0.2 μM), and radiation (10 Gy). Values represent the mean±SEM (n=4). Cytotoxic response is expressed as a percentage compared to cells receiving no chemotherapeutic agents or radiation exposure. FIG. 7E shows the effect of a small molecule inhibitor of SAT1 enzymatic activity (diminazene aceturate; DMA) in U251 cells (GBM), as compared to SAT1 knock-down (“LNP_siSAT1”), in the absence or presence of radiation (“10 Gy”). Coefficient of Therapeutic Interaction (CTI) is measured by the general formula AB/(AxB). CTI <1 represents a synergistic effect; CTI=1 represents an additive effect; and CTI >1 represents an antagonist effect. Control represents untreated U251 cells. FIG. 7F shows the results of a further experiment on U251 cells (40,000 cells seeded/well in 24-well plate treated with 80 nM LNP-siSCR (“Control) or LNP-siSAT1) using a higher dose of radiation (15 Gy at 28 h+15 Gy at 72 h; “15 Gy”).

FIG. 8 shows a representative single-cell alkaline gel electrophoresis (Comet Assay) in U251 cells. FIG. 8A shows U251 cells transfected with LNP-siSCR (control) 6 hours after irradiation (10 Gy).

FIG. 8A shows U251 cells transfected with LNP-siSAT1 (“SAT1_KD”) 6 hours after irradiation (10 Gy). The U251 cells were stained with SYBR green and observed under a fluorescent microscope. FIG. 8C shows the Extent Tail Moment (Tail DNA %× Length of tail) calculated from fifty individual cells.

FIG. 9A shows a representative immunofluorescence image of γ-H2AX (green) in LNP-siSCR transfected (“siCtrl”) and LNP-siSAT1 transfected (“siSAT1”) U251 cells, 6 hours after irradiation with 1 Gy. The bar graph sin FIG. 9B shows the average number of SAT1 foci counted from at least 30 individual cells.

FIG. 10 shows the result of an in vitro blood brain barrier (BBB) co-culture assay. FIG. 10A shows a schematic illustration of the in vitro BBB co-culture model showing the orientation of the BBB (hCMEC/D3) and glioblastoma (U251) cells. FIG. 10B shows the flux of the large molecular weight permeability marker IR-dye PEG across non-treated and ADTC5 and LNP_siSAT1 (in the donor compartment) treated hCMEC/D3 monolayers. FIG. 10C shows changes in SAT1 mRNA levels in U251 cells from co-culture model following treatment with LNP-siSAT1 with and without ADTC5 permeability enhancing peptide. The expression of SAT1 was determined following treatment. Values represent the mean±SEM (n=3).

FIG. 11A shows the results of three different cadherin peptides on hCMEC/d3 permeability for markers of different diameters. The effects of cadherin peptides (0.1 mM) on hCMEC/d3 monolayer permeability were examined using various fluorescent permeability markers in the BBB microfluidic model described in FIG. 11A. Permeability markers included sodium fluorescein (NaF, ˜0.9 nm), Irdye™ 800 CW (Irdye, ˜1.3 nm), fluorescein labeled dextran (FDX70KD, ˜5 nm), and lipid nanoparticles (LNP, ˜80 nm). FIG. 11B shows the fold-change in permeability of the same permeability markers with various cadherin peptides in hCMEC/d3 monolayers. Values represent mean±SEM of 3 microfluidic cells. *p<0.05; **p<0.01; ***p<0.001.

FIG. 12 shows the results of the permeability of lipid nanoparticles (LNP1-3) having different diameters in the BBB microfluidic model employed in FIG. 10A. Permeability coefficients for the different sized lipid nanoparticles with siSAT1 (LNP1-3) were determined under control conditions (media alone) or in the presence of 0.5 mM cadherin peptide ADTC5 to transiently open the blood-brain barrier. LNP1˜153.5 nm; LNP2˜103.4 nm; and LNP3˜98.2 nm. Values represent the mean±SEM of 3 microfluidic cells per treatment group. **p<0.01 and ****p<0.001 compared to LNP control as determined by ANOVA with multiple comparison of the means.

FIG. 13 shows the results of the permeability coefficients for LNP1 (˜153.5 nm) in the BBB microfluidic model employed in FIG. 10A. Cells were exposed to LNP1 alone (“No Peptide”) or various cadherin peptides (HAVN1, ADTC5, or ADT-N) to transiently increase permeability of brain endothelial cells. Values are the mean±SEM of 3 microfluidic units per treatment group. **p<0.01 compared to LNP1 control group (no peptide).

FIG. 14 shows the effect of treatment with N(1),N(11)-diethylnorspermine (DENSPM) (10 μM) on induction of SAT1 mRNA (FIG. 14A) and protein expression by Western blot (FIG. 14B) in U251 cells under uninduced (“control”) and induce (“DENSPM”) conditions (mean=+SD, n=3).

FIG. 15 shows the results of gene expression (FIGS. 15A and 15C) and protein expression (FIGS. 15B and 15D) of N-acetyltransferases NAT1 and NAT2 in U251 cells under control (uninduced) and induced conditions (DENSPM; 10 μM) (24 h and 48 h). (mean=+SD, n=3).

FIG. 16 shows the results of acetylated-Amantadine (Ac-Amantadine) concentration by LC-MS analysis (FIG. 16A) and activity of U251 cells (FIG. 16B) with or without induction in the presence of 100 μM of Amantadine in T175 multi-layer flask. (mean=+SD, n=3).

FIG. 17 shows the results of acetylated-rimantadine (Ac-Rimantadine) concentration by LC-MS analysis (FIG. 16A) and activity of U251 cells (FIG. 17B) with or without induction in the presence of 100 μM of Rimantadine in T175 multi-layer flask. FIG. 17C SAT1 induction was confirmed by Western blot analysis of the protein sample. (mean=+SD, n=3).

FIG. 18 shows the results of Ac-rimantadine production upon incubation of human recombinant enzymes (hr) SAT1, NAT1 and NAT2 with Rimantadine, as analyzed by LC-MS (mean=+SD, n=3).

FIG. 19 shows the results of the permeability of amantadine, rimantadine, and their acetylated metabolites in the BBB microfluidic model described in FIG. 10A. Permeability coefficients are the mean±SD of 3 microfluidic units.

### SEQUENCE LISTING

This application contains a Sequence Listing in computer readable form created Oct. 13, 2021. The computer readable form is incorporated herein by reference.

TABLE 1
Sequence Listing Description

| SEQ ID NO: | Description |
|---|---|
| 1 | siSAT sense oligo |
| | (CCAUCCAUCAACUUCUAUAtt) |
| 2 | siSAT antisense oligo |
| | (UAUAGAAGUUGAUGGAUGGtt) |
| 3 | SAT1 primer-sense |
| 4 | SAT1 primer-antisense |
| 5 | control 18S primer-sense |
| 6 | control 18S primer-antisense |

### DETAILED DESCRIPTION

In a first aspect, described herein is a biocompatible lipid nanoparticle (LNP) composition suitable for delivering RNA or other therapeutic payloads into cells and tissues of a subject. In some embodiments, the biocompatible LNP composition described herein comprises an ionizable cationic lipid as a core component to aid in the electrostatic loading of the RNA payload while reducing cell toxicity observed with conventional, non-cationic lipid formulations.

In some embodiments, the biocompatible LNP composition described herein may comprise, or consist essentially of, an RNA payload (e.g., siRNA) or other therapeutic payload encapsulated in a lipid component, the lipid component comprising a mixture of: (a) an ionizable cationic lipid (e.g., ionizable cationic unsaturated lipid) having a polar head group with a pKa of below 7; (b) a PEGylated lipid; (c) a sterol; and (d) a phospholipid.

As used herein, the expression “consisting essentially of” or “consists essentially of” refers to those elements required for a given embodiment. The expression permits the presence of additional elements that do not materially affect the basic and novel or functional characteristic(s) of that embodiment of the invention. In the context of LNP compositions described herein, the expressions “consisting essentially of” or “consists essentially of” refer to the elements required to achieve intracellular RNA payload delivery and for the RNA to exert its desired biological effect. For greater clarity, the expressions do not exclude the possibility that other additional non-essential ingredients (e.g., excipients, fillers, stabilizers, or inert components) that do not materially change the function or delivery properties of LNP compositions described herein.

In some embodiments, the biocompatible LNP compositions described herein are characterized by nanoparticles having a hydrodynamic size of about 50 to about 160 nm, about 50 to about 155 nm, about 50 to about 150 nm, about 50 to about 140 nm, about 50 to about 130 nm, about 60 to about 120 nm, about 60 to about 110 nm, about 60 to about 100 nm, about 65 to about 95 nm, about 70 to about 90 nm, about 75 to about 85 nm, or about 50, 55, 60, 65, 70, 75, 80, 85, 90, 95, 100, 110, 120, 130, 140, 150, 155, 160, 165, 170, or 175 nm. In some embodiments, the microfluidics-based biocompatible LNP compositions described herein are produced without an extrusion step through a filter, which is a necessary step in many conventional formulations to achieve their nanoparticle size.

In some embodiments, the biocompatible LNP compositions described herein are characterized by nanoparticles having a net neutral surface charge at physiological pH (e.g., zeta potential of below 0.6, 0.5, 0.4, 0.3, or 0.2). While nanoparticles with a net positive surface charge have been previously reported to potentially exhibit better cellular uptake in vitro than their neutral or negative surface charged counterparts, cationic nanoparticles in the context of systemic administration have the drawback of rapid clearance by nonspecific binding and phagocytosis. Thus, biocompatible LNP compositions described herein may exhibit a longer circulation half-life and have a better chance of accumulating in target cells/tissues than cationic LNPs.

In some embodiments, the biocompatible LNP compositions described herein are characterized by nanoparticles having a polydispersity index (PDI) of below about 0.3, 0.25, 0.2, 0.19, 0.18, 0.17, or 0.16. In some embodiments, such PDI values are attained without the need for an extrusion step through a filter.

In some embodiments, the biocompatible LNP compositions described herein have an N/P ratio (i.e., the ratio between cationic amines in the lipid component and the anionic phosphates on the RNA payload) of between 12 to 20, 13 to 19, 13 to 18, 13 to 17, or 14 to 16. In some embodiments, the biocompatible LNP compositions described herein have an N/P ratio of about 12, 13, 14, 15, 16, 17, 18, 19, or 20. Without being bound by theory, N/P ratios below a payload delivery lower limit may not deliver sufficient siRNA payload to achieve the desired level mRNA knockdown, while N/P ratios above a toxicity threshold upper limit may result in undesirable cytotoxicity. In some embodiments, the biocompatible LNP compositions described herein have an N/P ratio between a payload delivery lower limit and a toxicity threshold upper limit.

In some embodiments, the biocompatible LNP compositions described herein may comprise an ionizable cationic unsaturated lipid such as 1,2-dioleoyl-3-dimethylammonium-propane (DODAP, which has a pKa of 6.6-7); 1,2-dioleyloxy-N,N-dimethyl-3-aminopropane (DODMA); heptatriaconta-6,9,28,31-tetraen-19-yl 4-(dimethylamino) butanoate (DLin-MC3-DMA or MC3); 2,2-dilinoleyl-4-(2-dimethylaminoethyl)-[1,3]-dioxolane (DLin-KC2-DMA or KC2); or other pharmaceutically acceptable ionizable cationic unsaturated lipid; or any combination thereof. Ionizable cationic lipids carry a cationic charge at acidic pH and, therefore, can electrostatically bind to the negatively charged RNA payloads, which may explain the high encapsulation efficiency of siRNA shown herein in contrast to lower encapsulation efficiencies previous reported (e.g., Kulkarni et al., 2018A and Kulkarni et al., 2018B). In some embodiments, the ionizable cationic unsaturated lipid may constitute between 30 to 70, 35 to 65, 40 to 60, 47 to 57, 45 to 55%, or about 35, 40, 45, 50, 55, 60, 65, or 70%, of the lipid component of the biocompatible LNP compositions described herein.

In some embodiments, the biocompatible LNP compositions described herein may comprise a PEGylated lipid such as (1,2-distearoyl-sn-glycero-3-phosphorylethanolamine)-PEG (DSPE-PEG) or (1,2-dimyristoyl-rac-glycero-3-methoxy)-PEG (DMG-PEG). In some embodiments, the surface PEG-lipid groups may be beneficial for LNP formation, particle size, stability, and/or circulation half-life. In some embodiments, the size of the PEG moiety may be between 1K and 5K, 1.5K and 4.5K, 1.5K and 4K, 1.5K and 3.5K, 1.5K and 3K, or about 1K, 1.5K, 2K, 2.5K, 3K, 3.5K, 4K, 4.5K, or 5K.

In some embodiments, the biocompatible LNP compositions described herein may comprise a sterol such as cholesterol or other pharmaceutically acceptable sterol (e.g., plant or animal sterol).

In some embodiments, the biocompatible LNP compositions described herein may comprise a phospholipid such as distearoylphosphatidylcholine (DSPC); 1,2-dioleoyl-sn-glycero-3-phosphocholine (DOPC); or 1-palmitoyl-2-oleoyl-glycero-3-phosphocholine (POPC). In some embodiments, a less rigid phospholipid such as DSPC may be considered to enable tighter packing and smaller nanoparticle sizes.

In some embodiments, the biocompatible LNP compositions described herein may comprise or consist essentially of an RNA payload encapsulated in an ionizable lipid/PEGylated lipid/sterol/phospholipid mixture (e.g., DODAP/DSPE/cholesterol/DSPC lipid mixture), such as at a molar ratio of about 50/10/37.5/1.5, respectively. Without being bound by theory, at an initial stage of LNP formation, small clusters containing siRNA and closely opposed cationic lipids are believed to be formed, which may then fuse and grow until DSPC and cholesterol sequester and arrest the growth. During LNP formation, the PEGylated lipids assemble along the surface, providing steric stabilization. The surface sequestration of neutral DSPC/cholesterol followed by PEGylated lipids may explain the neutral zeta potential of the LNP-siRNA formulations described herein. Having a neutral surface charge is advantageous as it may help evade nonspecific binding and detection by the mononuclear phagocyte system.

In some embodiments, the RNA payload described herein may comprise or consist essentially of siRNA or a mixture of siRNAs. In some embodiments, the RNA payload described herein may comprise or consist essentially of siRNAs or a mixture of siRNAs for inhibiting expression of Spermidine/spermine N1-acetyltransferase 1 (SAT1).

As used herein, the expression “siRNA”, or small-interfering RNA, refers to short double-stranded RNA molecules that target a certain gene and reduce or inhibit the expression of that gene, and eventual protein expression. In some embodiments, the siRNA has a sequence length of about 15-40 base pairs, preferably between 20-30 base pairs. In some embodiments, siRNAs do not include small hairpin RNAs (shRNAs), which are typically 80 base pair in length and form hairpin structures.

Described herein is an siRNA that is specific for a gene encoding SAT1 (SSAT1). However, any siRNA that targets any portion of the SAT1 gene may be encompassed herein. The siRNA may bind the SAT1 mRNA and inhibit/decrease its expression and/or inhibit its translation into a functional protein.

In some embodiments, the biocompatible LNP compositions described herein may be prepared by a method comprising microfluidic mixing of suitable volumes of an aqueous phase and an organic phase, the aqueous phase comprising the siRNA dissolved in an acidic buffer (e.g., acetate buffer such as pH 4), and the organic phase comprising the lipid component ingredients dissolved in a suitable alcohol such as ethanol, followed by dilution in a buffer at physiologic pH (e.g., pH between 7.2 and 8.0, 7.2 and 7.9, 7.2 and 7.8, 7.2 and 7.7, 7.2 and 7.6, 7.2 and 7.5, 7.2 and 7.4).

In some embodiments, the biocompatible LNP compositions described herein are for use in delivering the RNA payload (e.g., siRNA) to brain cells (e.g., brain tumor cells, preferably brain tumor cells characterized by SAT1 overexpression in comparison to corresponding non-tumor cells).

In some embodiments, the expression “brain tumor cells” as used herein refers to one or more cells in a tumor located anywhere in the brain or the central nervous system. In some embodiments, brain tumor cells as used herein may refer to brain tumor cell lines, cells from one or more tumors biopsied or extracted from a mammal (e.g., human or mouse), or tumor cells in a tumor located in the brain or CNS of a mammal. In some embodiments, the brain tumor cells may be tumor cells from any brain or CNS cancer such as but not limited to carcinoma, adenoma, neuroma, acoustic neuroma, astrocytoma, brain metastases, choroid plexus carcinoma, craniopharyngioma, embryonal tumors, ependymoma, glioblastoma, glioma, medulloblastoma, meningioma, oligodendroglioma, pediatric brain tumors, pineoblastoma, or pituitary tumors. In some embodiments, “brain tumor cells” as used herein may refer to a glioblastoma that is a high-grade or low-grade glioma, or any one of grades 1˜4 gliomas. In some embodiments, the glioblastoma may be an isocitrate dehydrogenase (IDH)-wildtype or -mutant glioma. In some embodiments the glioma/glioblastoma may comprise other known genetic mutations, such as but not limited to MGMT, TERT, TP53, ATRX, PDGFRA, NF1 EGFR, NEFL, GABRAI, SYT1, SLC12A5, RB, PI3K/AKT and PTEN.

In some embodiments, the biocompatible LNP compositions described herein are for use in systemic or intravenous delivery with a blood-brain barrier permeabilizing agent (e.g., a cadherin binding peptide, such as a linear or cyclic ADTC5, HAVN1, HAVN2, ADTHAV, HAV6, HAV4, CHAVc1, or cHAVc3 peptide). Cadherin binding peptides are believed to increase blood-brain barrier permeability via short, reversible opening of the intercellular junctions controlling paracellular diffusion of solutes (On et al., 2014). The cadherin peptides generally bind to the EC domain of E-cadherin, a membrane protein of the adherens junction of the blood-brain barrier. The peptide-E-cadherin binding inhibits the cadherin-cadherin homodimer interactions between adjacent brain capillary endothelial cells resulting in the disruption of the blood-brain barrier tight junction.

In some embodiments, the biocompatible LNP compositions described herein are for use in the manufacture of a medicament for treating a disease or disorder that is ameliorated by inhibiting expression of the gene targeted by siRNA payloads described herein. In some embodiments, the biocompatible LNP compositions described herein are for use in the manufacture of a medicament for inhibiting the growth of brain tumor cells (e.g., glioblastoma or other brain tumor cells as described herein).

In a further aspect, described herein is a method for inhibiting the growth of brain tumor cells, the method comprising contacting the brain tumor cells with a biocompatible lipid nanoparticle composition comprising an siRNA for inhibiting expression of Spermidine/spermine N1-acetyltransferase 1 (SAT1), encapsulated in a lipid component. In some embodiments, the brain tumor cells are glioblastoma cells (e.g., glioblastoma cells characterized by overexpression of SAT1 in comparison to corresponding non-cancer cells). In some embodiments, the lipid component, the nanoparticles, and/or the biocompatible LNP composition are as described herein.

In some embodiments, the methods described herein are for inhibiting the growth of brain tumor cells in a subject to be treated and the method comprises administering a biocompatible lipid nanoparticle composition described herein comprising an siRNA for inhibiting expression of Spermidine/spermine N1-acetyltransferase 1 (SAT1) directly to brain tissue (e.g., via intracranial injection, or intratumoral injection), thereby bypassing the blood-brain barrier. In some embodiments, the biocompatible LNP compositions described herein may formulated in slow-release formulation (e.g., hydrogel) in implanted or administered directly to the tissue of a patient (e.g., following tumor resection).

In some embodiments, the methods described herein are for inhibiting the growth of brain tumor cells in a subject to be treated and the method comprises administering to a biocompatible lipid nanoparticle composition described herein comprising an siRNA for inhibiting expression of Spermidine/spermine N1-acetyltransferase 1 (SAT1) intravenously in combination with a blood-brain barrier permeabilizing agent. In some embodiments, the blood-brain barrier permeabilizing agent is a cadherin binding peptide, such as a peptide derived from the extracellular-1 (EC-1) domain of E-cadherin. In some embodiments, the blood-brain barrier permeabilizing agent is a cadherin binding peptide derived from the bulge region (HAV peptides) or groove region (ADT peptides) from E-cadherin EC-1 domain, combinations thereof (e.g., ADTHAV peptides), or variants thereof (Ulapane et., 2019a and Ulapane et., 2019b). In some embodiments, the ADT peptides described herein may comprise a peptide derived from the C-terminal region (e.g., ADTC5 or HAVN1) or N-terminal region of the EC-1 domain of E-cadherin. In some embodiments, cadherin binding peptides described herein may comprise a linear or cyclic ADTC5, HAVN1, HAVN2, HAV6, HAV4, CHAVc1, cHAVc3, ADTHAV peptides, combinations or variants thereof. In some aspects, the cadherin peptides described herein may include those described in WO2020257745A1, and are herein incorporated by reference in their entirety.

In some embodiments, the method for inhibiting the growth of brain tumor cells in subject described herein comprise administering a lipid nanoparticle composition that inhibits expression of SAT1 as described herein, in combination with a chemotherapy and/or radiation therapy. In some embodiments, the chemotherapy may comprise an alkylating agent (e.g., carmustine, temozolomide), a topoisomerase inhibitor (e.g., topotecan), an anthracycline (e.g., doxorubicin), or any combination thereof. In some embodiments, the chemotherapy may lack an anthracycline (e.g., doxorubicin). In some embodiments, radiation therapy may include radiation with at least 1, 2, 3, 4, 5, 6, 7, 8, 9, 10, 11, 12, 13, 14, 15, 16, 17, 18, 19, or 20 Gy of radiation. In some embodiments, treatment of brain tumor cells with the SAT1 inhibiting biocompatible LNP compositions described herein may decreases the dosage or frequency of chemotherapy and/or radiation normally required for treatment.

In some embodiments, inhibiting the growth of brain tumor cells may include reducing the size of the brain tumor (as compared to before treatment), inhibiting or reducing proliferation of the brain tumor cells, and/or inhibiting or reducing metastasis of the brain tumor to other parts of brain, CNS, and/or other tissues. The siRNA may be administered or given at any dose effective or sufficient to reduce the size of the brain tumor (as compared to before treatment), inhibit or reduce proliferation of the brain tumor cells, and/or inhibit or reduce metastasis of the brain tumor to other parts of brain, CNS, and/or other tissues.

In a further aspect, described herein is a cadherin binding peptide for use in improving the delivery of RNA (e.g., siRNA) or other therapeutic cargo encapsulated in a biocompatible lipid nanoparticle composition as described herein. In a further aspect, described herein is a method for increasing the delivery of an RNA payload across the blood-brain barrier of a subject, the method comprising: providing a biocompatible lipid nanoparticle composition comprising the RNA payload encapsulated therein (e.g., a biocompatible LNP composition as described herein); administering the lipid nanoparticle composition intravenously to the subject in combination with cadherin binding peptide that transiently increases blood-brain barrier permeability. In some embodiments, the cadherin binding peptide may be or comprise a linear or cyclic ADTC5, HAVN1, HAVN2, ADTHAV, HAV6, HAV4, CHAVc1, or cHAVc3 peptide.

In some embodiments, the biocompatible LNP compositions described herein may be administered with a blood-brain barrier permeabilizing agent other than a cadherin binding peptide. Osmotic (hypertonic mannitol), and pharmacological (Bradykynin Analogs, alkylglycerols, lysophosphatidic acid) strategies lead to disruption of the blood-brain barrier and may enhance paracellular permeability of biocompatible LNP compositions described herein, although the effects on blood-brain barrier permeability may be more prolonged.

In some aspects, described herein is a method for identifying a subject having glioblastoma, wherein said method comprises: (a) determining the expression level and/or activity level of Spermidine/spermine N1-acetyltransferase 1 (SAT1) in a biological sample from a subject clinically assessed as having or suspected of having glioblastoma; and (b) identifying the subject having glioblastoma when the SAT1 expression and/or activity level is elevated with respect to a control sample (e.g., healthy control, or a patient not having glioblastoma).

In some aspects, described herein is a method for the treatment of glioblastoma in a glioblastoma subject in need thereof, wherein said method comprises: (a) determining the expression level and/or activity level of Spermidine/spermine N-acetyltransferase 1 (SAT1) in a biological sample from a subject clinically assessed as having or suspected of having glioblastoma; (b) identifying the subject having glioblastoma when the SAT1 expression and/or activity level is elevated with respect to that of a control sample (e.g., healthy control, or a patient not having glioblastoma); and (c) when the glioblastoma subject is identified, treating glioblastoma subject with anti-glioblastoma therapy (e.g., radiation, surgery, immunotherapy, and/or chemotherapy).

In some aspects, described herein is a method for diagnosing or determining a progression/severity of glioblastoma in a subject, wherein said method comprises: (a) determining the expression level and/or activity level of Spermidine/spermine N1-acetyltransferase 1 (SAT1) in a biological sample from a subject clinically assessed as having or suspected of having glioblastoma; and (b) diagnosing the subject as having glioblastoma or determining the progression or severity of glioblastoma by observing significantly increased SAT1 expression level and/or activity level of SAT1 as compared to that indicative of a subject not having glioblastoma.

In some aspects, described herein is a method for clinically assessing glioblastoma in a human subject having or suspected of having glioblastoma, the method comprising: (a) providing a biological sample from the subject; (b) determining the expression level and/or activity level of Spermidine/spermine N1-acetyltransferase 1 (SAT1) in said biological sample; and (c) clinically assessing glioblastoma in the subject by comparing the expression and/or activity levels of SAT1 to a suitable reference value indicative of the presence, stage, and/or progression of glioblastoma.

In some embodiments, the step of determining the expression level and/or activity level of SAT1 in said biological sample may comprise: determining the level of one or more corresponding metabolites of substrates of SAT1 in said sample; determining the level of one or more acetylated substrates of SAT1 in said sample; and/or determining the levels of acetylated-amantadine, acetylated-rimantadine, and/or acetylated-tocainide in said sample.

In some embodiments, the above-mentioned methods may further comprise administering one or more substrates of SAT1 to said subject prior to the step of determining the expression level and/or activity level of SAT1 in the biological sample. In some embodiments, the above-mentioned methods may further comprise administering amantadine, rimantadine, and/or tocainide to said subject prior to the step of determining the expression level and/or activity level of SAT1 in the biological sample.

In some aspects, described herein is a method for producing or modifying a glioblastoma testing program or a test for detecting or clinically assessing glioblastoma in a subject, the method comprising adding or integrating into said program or test quantifying in a biological sample (e.g., blood sample, urine sample, saliva sample) from a subject clinically assessed as having or suspected of having glioblastoma, the level of an acetylated substrate of SAT1 (e.g., acetylated-amantadine, acetylated-rimantadine, and/or acetylated-tocainide). As used herein, the expression “glioblastoma testing program” refers to a clinical multi-faceted glioblastoma testing program in which a medical professional takes into consideration a number of factors to assess a patient's likelihood of glioblastoma, such as a patient's symptoms, history, other complementary investigations such as imaging and biopsy results. Thus, a patient “clinically assessed as having or suspected of having glioblastoma” is expected to have preexisting factors that would prompt a medical professional to consider quantifying the levels of the acetylated SAT1 substrates described herein.

In some embodiments, the subject described herein has been previously administered with one or more substrates of SAT1 (e.g., amantadine, rimantadine, and/or tocainide) prior to obtaining the sample. In some embodiments, the biological sample described herein may be a blood sample, serum sample, plasma sample, urine sample, a tissue sample, a biopsy sample, or a tumor sample (e.g., a brain tumor sample). In some embodiments, the methods described herein may be an in vitro, ex vivo, and/or in vivo method.

In some aspects, described herein is a kit for use in diagnosing or determining the progression/severity of glioblastoma in a subject, said kit comprising one or more reagents for determining the expression level and/or activity level of Spermidine/spermine N1-acetyltransferase 1 (SAT1) in a biological sample from the subject. In some embodiments, the kit comprises one or more reagents for determining the levels of one or more corresponding metabolites of substrates of SAT1. In some embodiments, the kit comprises one or more reagents for determining the levels of acetylated-amantadine, acetylated-rimantadine, and/or acetylated-tocainide in said sample. In some embodiments, the biological sample is a blood sample, serum sample, plasma sample, urine sample, a tissue sample, a biopsy sample, or a tumor sample (e.g., a brain tumor sample). In some embodiments, the subject has been previously administered with one or more substrates of SAT1 (e.g., amantadine, rimantadine, and/or tocainide) prior to obtaining the sample.

### Items

1. A biocompatible lipid nanoparticle composition comprising, or consisting essentially of, an siRNA encapsulated in a lipid component, the lipid component comprising a mixture of: (a) an ionizable cationic lipid (e.g., ionizable cationic unsaturated lipid) having a polar head group with a pKa of below 7; (b) a PEGylated lipid; (c) a sterol; and (d) a phospholipid.
2. The biocompatible lipid nanoparticle composition of item 1, wherein the nanoparticles have: (i) a hydrodynamic size of about 60 to about 160 nm, about 60 to about 155 nm, about 60 to about 150 nm, about 65 to about 95 nm, about 70 to about 90 nm, about 75 to about 85 nm, or about 80 nm; (ii) a net neutral surface charge at physiological pH (e.g., zeta potential of below 0.6, 0.5, 0.4, 0.3, or 0.2); (iii) a polydispersity index (PDI) of below 0.2; or (iv) an N/P ratio of between 12 to 20, 13 to 19, 13 to 18, 13 to 17, 14 to 16, or about 12, 13, 14, 15, 16, 17, 18, 19, or 20; (v) any combination of (i) to (iii).
3. The biocompatible lipid nanoparticle composition of item 1 or 2, wherein: (a) the ionizable cationic unsaturated lipid is: 1,2-dioleoyl-3-dimethylammonium-propane (DODAP); 1,2-dioleyloxy-N,N-dimethyl-3-aminopropane (DODMA); heptatriaconta-6,9,28,31-tetraen-19-yl 4-(dimethylamino) butanoate (DLin-MC3-DMA or MC3); 2,2-dilinoleyl-4-(2-dimethylaminoethyl)-[1,3]-dioxolane (DLin-KC2-DMA or KC2); or other pharmaceutically acceptable ionizable cationic unsaturated lipid; or any combination thereof; (b) the PEGylated lipid is (1,2-distearoyl-sn-glycero-3-phosphorylethanolamine)-PEG (DSPE-PEG); (1,2-dimyristoyl-rac-glycero-3-methoxy)-PEG (DMG-PEG); or other pharmaceutically suitable PEGylated lipid; (c) the sterol is cholesterol or other pharmaceutically suitable sterol; (d) the phospholipid is distearoylphosphatidylcholine (DSPC); 1,2-dioleoyl-sn-glycero-3-phosphocholine (DOPC); 1-palmitoyl-2-oleoyl-glycero-3-phosphocholine (POPC); or other pharmaceutically suitable phospholipid; or (e) any combination of (a) to (d).
4. The biocompatible lipid nanoparticle composition of item 3, wherein the lipid component comprises a molar ratio of between 30 to 70, 35 to 65, 40 to 60, 47 to 57, 45 to 55%, or about 35, 40, 45, 50, 55, 60, 65, or 70% of the ionizable cationic unsaturated lipid.
5. The biocompatible lipid nanoparticle composition of any one of items 1 to 4, wherein the siRNA comprises an siRNA for inhibiting expression of Spermidine/spermine N1-acetyltransferase 1 (SAT1).
6. The biocompatible lipid nanoparticle composition of any one of items 1 to 5, which is prepared by microfluidic mixing of suitable volumes of an aqueous phase and an organic phase, the aqueous phase comprising the siRNA dissolved in an acidic buffer (e.g., acetate buffer), and the organic phase comprising the lipid component ingredients dissolved in ethanol, followed by dilution in a buffer at physiologic pH.
7. The biocompatible lipid nanoparticle composition of any one of items 1 to 6 for use in delivering the siRNA payload to brain cells (e.g., brain tumor cells, preferably brain tumor cells characterized by SAT1 overexpression in comparison to corresponding non-tumor cells).
8. The biocompatible lipid nanoparticle composition of any one of items 1 to 6 for use in intravenous delivery with a blood-brain barrier permeabilizing agent (e.g., a cadherin binding peptide, such as a linear or cyclic ADTC5, HAVN1, HAVN2, ADTHAV, HAV6, HAV4, cHAVcl, or cHAVc3 peptide).
9. A method for inhibiting the growth of brain tumor cells, the method comprising contacting the brain tumor cells with a biocompatible lipid nanoparticle composition comprising an siRNA for inhibiting expression of Spermidine/spermine N1-acetyltransferase 1 (SAT1), encapsulated in a lipid component.
10. The method of item 9, wherein the brain tumor cells are grade 1, 2, 3 and/or 4 brain tumors, gliomas, astrocytomas, glioblastoma cells (e.g., glioblastoma cells characterized by overexpression of SAT1; highly proliferative glioblastoma cells; glioblastoma cells deficient in DNA damage repair mechanisms; glioblastoma cells resistant to temozolomide (TMZ)).
11. The method of item 9 or 10, wherein the lipid component is as defined in item 1 and/or the nanoparticles are as defined in item 2.
12. The method of any one of items 9 to 11, wherein the lipid nanoparticle composition is as defined in any one of items 1 to 6.
13. The method of any one of items 9 to 12, where the brain tumor cells are in a subject to be treated and the contacting is performed by administering the lipid nanoparticle composition in the brain of the subject, thereby bypassing the blood-brain barrier.
14. The method of any one of items 9 to 12, where the brain tumor cells are in a subject to be treated and the lipid nanoparticle composition is administered intravenously in combination with a blood-brain barrier permeabilizing agent.
15. The method of item 14, wherein the blood-brain barrier permeabilizing agent is a cadherin binding peptide (e.g., a linear or cyclic ADTC5, HAVN1, HAVN2, ADTHAV, HAV6, HAV4, CHAVc1, or cHAVc3 peptide).
16. The method of any one of items 13 to 15, wherein the lipid nanoparticle composition is administered in combination with a chemotherapy and/or radiation.
17. The method of item 16, wherein the chemotherapy comprises an alkylating agent (e.g., carmustine, temozolomide), a topoisomerase inhibitor (e.g., topotecan), an anthracycline (e.g., doxorubicin), or any combination thereof.
18. The method of item 16 or 17, wherein the chemotherapy lacks an anthracycline (e.g., doxorubicin).
19. A cadherin binding peptide for use in improving the delivery (e.g., across the blood-brain barrier) of RNA (e.g., siRNA), or another therapeutic cargo, encapsulated in a biocompatible lipid nanoparticle composition.
20. The cadherin binding peptide for use of item 19, wherein the biocompatible lipid nanoparticle composition is as defined in any one of items 1 to 11.
21. The cadherin binding peptide for use of item 19 or 20, wherein the cadherin binding peptide is or comprises linear or cyclic ADTC5, HAVN1, HAVN2, ADTHAV, HAV6, HAV4, CHAVc1, or cHAVc3.
22. A method for increasing the delivery of an RNA payload across the blood-brain barrier of a subject, the method comprising: providing a biocompatible lipid nanoparticle composition comprising the RNA payload encapsulated therein; administering the lipid nanoparticle composition intravenously to the subject in combination with cadherin binding peptide that transiently increases blood-brain barrier permeability.
23. The method of item 22, wherein the biocompatible lipid nanoparticle composition is as defined in any one of items 1 to 11, and/or the cadherin binding peptide is or comprises linear or cyclic ADTC5, HAVN1, HAVN2, ADTHAV, HAV6, HAV4, CHAVc1, or cHAVc3.
24. A method for identifying a subject having glioblastoma, wherein said method comprises: (a) determining the expression level and/or activity level of Spermidine/spermine N1-acetyltransferase 1 (SAT1) in a biological sample from a subject clinically assessed as having or suspected of having glioblastoma; and (b) identifying the subject having glioblastoma when the SAT1 expression and/or activity level is elevated with respect to a control sample (e.g., healthy control, or a patient not having glioblastoma).
25. A method for the treatment of glioblastoma in a glioblastoma subject in need thereof, wherein said method comprises: (a) determining the expression level and/or activity level of Spermidine/spermine N1-acetyltransferase 1 (SAT1) in a biological sample from a subject clinically assessed as having or suspected of having glioblastoma; (b) identifying the subject having glioblastoma when the SAT1 expression and/or activity level is elevated with respect to that of a control sample (e.g., healthy control, or a patient not having glioblastoma); and (c) when the glioblastoma subject is identified, treating glioblastoma subject with anti-glioblastoma therapy (e.g., radiation, surgery, immunotherapy, and/or chemotherapy).
26. A method for diagnosing or determining a progression/severity of glioblastoma in a subject, wherein said method comprises: (a) determining the expression level and/or activity level of Spermidine/spermine N1-acetyltransferase 1 (SAT1) in a biological sample from a subject clinically assessed as having or suspected of having glioblastoma; and (b) diagnosing the subject as having glioblastoma or determining the progression or severity of glioblastoma by observing significantly increased SAT1 expression level and/or activity level of SAT1 as compared to that indicative of a subject not having glioblastoma.
27. A method for clinically assessing glioblastoma in a human subject having or suspected of having glioblastoma, the method comprising: (a) providing a biological sample from the subject; (b) determining the expression level and/or activity level of Spermidine/spermine N1-acetyltransferase 1 (SAT1) in said biological sample; and (c) clinically assessing glioblastoma in the subject by comparing the expression and/or activity levels of SAT1 to a suitable reference value indicative of the presence, stage, and/or progression of glioblastoma.
28. The method of any one of items 24 to 27, wherein the step of determining the expression level and/or activity level of SAT1 in said biological sample comprises determining the level of one or more corresponding metabolites of substrates of SAT1 in said sample.
29. The method of any one of items 24 to 28, wherein the step of determining the expression level and/or activity level of SAT1 in said biological sample comprises determining the level of one or more acetylated substrates of SAT1 in said sample.
30. The method of any one of items 24 to 29, wherein the step of determining the expression level and/or activity level of SAT1 in said biological sample comprises determining the levels of acetylated-amantadine, acetylated-rimantadine, and/or acetylated-tocainide in said sample.
31. The method of any one of items 24 to 30, further comprising administering one or more substrates of SAT1 to said subject prior to the step of determining the expression level and/or activity level of SAT1 in the biological sample.
32. The method of any one of items 24 to 31, further comprising administering amantadine, rimantadine, and/or tocainide to said subject prior to the step of determining the expression level and/or activity level of SAT1 in the biological sample.
33. A method for producing or modifying a glioblastoma test or a test for detecting glioblastoma, the method comprising adding or integrating into said test quantifying a panel of metabolites in a biological sample from a subject having or suspected of having glioblastoma, the panel comprising one or more corresponding metabolites of substrates of Spermidine/spermine N1-acetyltransferase 1 (SAT1).
34. The method of item 33, wherein the substrates of SAT1 are or comprise amantadine, rimantadine, and/or tocainide, and the corresponding metabolites are or comprise acetylated-amantadine, acetylated-rimantadine, and/or acetylated-tocainide.
35. The method of any one of items 24 to 30, 33, and 34, wherein the subject has been previously administered with one or more substrates of SAT1 (e.g., amantadine, rimantadine, and/or tocainide) prior to obtaining the sample.
36. The method of any one of items 24 to 35, wherein the biological sample is a blood sample, serum sample, plasma sample, urine sample, a tissue sample, a biopsy sample, or a tumor sample (e.g., a brain tumor sample).
37. The method of any one of items 24 to 36, wherein the method is an in vitro, ex vivo, and/or in vivo method.
38. A kit for use in diagnosing or determining the progression/severity of glioblastoma in a subject, said kit comprising one or more reagents for determining the expression level and/or activity level of Spermidine/spermine N1-acetyltransferase 1 (SAT1) in a biological sample from the subject.
39. The kit for use of item 38, wherein the kit comprises one or more reagents for determining the levels of one or more corresponding metabolites of substrates of SAT1.
40. The kit for use of item 38 or 39, wherein the kit comprises one or more reagents for determining the levels of acetylated-amantadine, acetylated-rimantadine, and/or acetylated-tocainide in said sample.
41. The kit for use of any one of items 38 to 40, wherein the biological sample is a blood sample, serum sample, plasma sample, urine sample, a tissue sample, a biopsy sample, or a tumor sample (e.g., a brain tumor sample).
42. The kit for use of any one of items 38 to 41, wherein the subject has been previously administered with one or more substrates of SAT1 (e.g., amantadine, rimantadine, and/or tocainide) prior to obtaining the sample.

### EXAMPLES

### Example 1: Materials and Methods

### 1.1 Materials

1,2-dioleoyl-3-dimethylammonium-propane (DODAP), 1,2-distearoyl-sn-glycero-3-phosphocholine (DSPC), and 1,2-distearoyl-sn-glycero-3-phosphoethanolamine-N-[(polyethylene glycol)-2000] (DSPE-PEG) were obtained from Avanti (Alabaster, AL), cholesterol, N-(3-Dimethylaminopropyl)-N′-ethylcarbodiimide hydrochloride (EDC) and N-hydroxysuccinimide (NHS) was obtained from Sigma-Aldrich (St. Louis, MO). 1,1′-Dioctadecyl-3,3,3′,3′-Tetramethylindotricarbocyanine Iodide (DiR) was obtained from Invitrogen (Carlsbad, CA). Recombinant human Apolipoprotein E (APOE) was purchased from Abcam (Toronto, ON).

### 1.2 Cell Culture

Human GBM cell line U251MG was grown in Dulbecco's Modified Eagle Medium: Nutrient Mixture F-12 (DMEM/F12) (Gibco, Carlsbad, CA) with 10% fetal bovine serum (FBS; Gibco), 1% penicillin-streptomycin (Gibco). The human brain microvascular endothelial cell line, hCMEC/D3 was obtained from Pierre-Oliver Couraud, INSERM, France. The cells (Passage 27-35) were cultured in EBM-2 (Lonza) media supplemented with 5% heat-inactivated FBS (Gibco), 1% penicillin-streptomycin (Gibco Carlsbad, CA), 1.4 μM hydrocortisone (Sigma), 5 g/mL ascorbic acid (Sigma), 1% lipid concentrate (Invitrogen), 10 mM HEPES (Gibco), and 1 ng/mL basic fibroblast growth factor (Gibco). Primary human astrocytes were grown in HA growth medium (CELL Applications) supplemented with 1% FBS and 1% penicillin-streptomycin. The murine macrophage cell line, ANA-1, was grown in Dulbecco's Modified Eagle Medium (DMEM) supplemented with 10% FBS 1% penicillin-streptomycin. For routine culture, all cells were grown in T75 flasks maintained at 37° C. in a humidified incubator with 5% CO₂.

### 1.3 Formulation of siRNA Encapsulated LNP

Appropriate volumes of lipids from individual stocks were mixed and diluted in ethanol, adhering to the molar ratio of DODAP (1,2-dioleoyl-3-dimethylammonium-propane)/DSPC (Distearoylphosphatidylcholine)/cholesterol/DSPE (1,2-Distearoyl-sn-glycero-3-phosphorylethanolamine)-PEG/Dioctadecyl-3,3,3 3 Tetramethylindocarbocyanine Perchlorate (DiIC18) of 50/10/37.5/1.5/1% and a total lipid concentration of 10 mg/mL. siRNA (SAT1 siRNAs: SEQ ID NOs: 1 and 2; control siRNAs: Silencer™ Select Negative Control No. 1 siRNA) was dissolved in sodium acetate buffer (25 mmol, pH=4) to yield 0.33 mg/mL concentration. For the microfluidic mixing, 1× volume of the lipid organic phase and 3× volumes of the siRNA aqueous phase were micromixed using a NanoAssembler™ Benchtop instrument (Precision NanoSystem, Vancouver, BC). A flow rate ratio (FRR) of 1:3 (organic: aqueous) and a total flow rate (TFR) of 12 mL/min was used to yield LNP-siRNA of about 80 nm diameter. The LNP-siRNA solution was diluted (50-times) in PBS (pH=7.4) and concentrated using a centrifugal filter (2000×g, MWCO 3000) to its original volume. Post-microfluidic formulation the crude LNP-siRNA products were incubated at room temperature for at least one hour. For the first wash, the crude products were then concentrated (¼ times or the equivalent volume of the organic phase) using a centrifugal filter (2000×g, MWCO 3000) and diluted (4×) using sodium acetate buffer (25 mmol, pH=4). The LNP product in sodium acetate buffer was further concentrated to the equivalent volume of the organic phase. The product from the first wash was then diluted (50×) in PBS (pH=7.4) and concentrated (2000×g, MWCO 3000) to the original volume to yield the final product. The concentration of the encapsulated siRNA in the LNP-siRNA formulation was measured using Thermo Scientific NanoDrop™ spectrophotometer (Madison, WI).

### 1.4 Size and Charge Determination of LNPs

The particle size (hydrodynamic) and the net surface charge (zeta potential) was measured using ZetaPALS™ (Brookhaven Instruments, NY, USA) dynamic light scattering instrument. Purified LNP formulations were diluted to 20 μg/mL lipid concentration in PBS (pH 7.4). The polydispersity index (PDI) obtained from the dynamic light scattering instrument was used to determine the size distribution of the LNPs (lower PDI meaning monodisperse LNPs).

### 1.5 Transfection Using LNP-siSAT1

For transfection, cells were seeded (20,000 cells/cm2) in T-25 flasks and grown to 70% confluency. The day before transfection, the complete media was replaced with DMEM/F-12 without FBS and antibiotics. LNP-siSAT1 (7 mL with 1 μg/mL APOE; 80 nM final siRNA concentration in transfection media) was added to the flask and incubated overnight in a CO₂ incubator at 37° C. The next day, the treatment was replaced with complete media, and the cells were placed in the CO₂ incubator. The knockdown of SAT1 at the mRNA and protein levels were determined at 48 and 72 hours after transfection, respectively, as described below.

### 1.6 Real-Time One-Step RT-PCR

Total mRNA was isolated from cells using TRIzol™ reagent (Invitrogen, Burlington, ON), following the manufacturer's protocol. The concentration of isolated mRNA in solution was estimated spectrophotometrically. The one-step qPCR reactions were performed using iTaq™ Universal SYBR™ Green One-Step Kit (Bio-Rad) following the manufacturer's protocol. For a 20 μL reaction, 0.5 μg mRNA was used. The primers (Invitrogen) specific for SAT1 (sense 5′-CTCCGGAAGGACACAGCATT-3′ [SEQ ID NO: 3] and antisense, 5′-ACCTCATTGCAACCTGGCTTA-3 ‘[SEQ ID NO: 4]) and the internal control 18S (sense 5’-AAACGGCTACCACATCCAAG-3′ [SEQ ID NO: 5] and antisense, 5′-CCTCCAATGGATCCTCGTTA-3′ [SEQ ID NO: 6]) were used. Thermocycling [Reverse transcription: 50° C. (10 min.), Polymerase activation and DNA denaturation: 95° C. (1 min.), 40 cycles (Denaturation: 95° C. (15 sec.), Annealing: 60° C. (60 sec.) and readout)] was carried out using Applied Biosystems™ AB7500 instrument. The relative mRNA levels compared to controls were calculated following the 2-AACT method (Schmittgen et al., 2008).

### 1.7 Western Blot

Cells were washed with PBS (3×) and lysed using RIPA buffer. Lysates in ice were sonicated for 10 seconds and centrifuged at 15000 g for fifteen minutes. The supernatant was collected, and the total protein concentration was measured using the Pierce™ BCA Protein Assay Kit (Fisher Scientific, Waltham, MA). The lysates (40 μg protein/well) were mixed with 5× loading buffer and separated on 10% polyacrylamide gel and subsequently transferred to polyvinylidene difluoride (PVDF) membranes. The PVDF membrane was incubated for an hour in blocking buffer [5% (w/v) skimmed milk in TBST-tris-buffered saline (pH 7.4) with 0.1% (v/v) Tween™ 20] for an hour at room temperature. The membrane was incubated overnight with anti-SAT1 antibody (2 μg/mL, PA1-16992) in 5% (w/v) skimmed milk in TBST-tris-buffered saline (pH 7.4) at 4° C. overnight. The membrane was washed (4×15 minutes) in TBST buffer and incubated with HRP-conjugated secondary antibody for one hour at room temperature. Later, the membranes were washed (4× 15 minutes) in TBST buffer and blots were visualized by enhanced chemoluminescence (Biorad) as per the manufacturer's protocol.

### 1.8 Cell Viability/Proliferation Assay

Cells were transfected in T-25 flask as discussed above. Forty-eight hours after transfection, the cells were seeded in 96-well plates (5000 cells/well). After 24-hours, the cells were exposed to no treatment or either 10 Gy radiation (RS-2000, Rad Source Technologies, Inc., Buford, GA, USA) or anticancer drugs carmustine (i.e., BCNU™ or BiCNU™) (100 μM), Doxorubicin (0.1 μM) and Topotecan (0.2 μM). After 24 hours, the media and treatments were removed and replaced with fresh media and cultured for 48 hours. The percentage of viable cells was determined using MTT assay (Norouzi et al, 2020).

### 1.9 Comet Assay

The comet assay was performed using a kit (Abcam) following the manufacturer's protocol. The assay on control and SAT KD (knock down) and irradiated U251 cells was performed six hours after irradiation exposure. The comet assay involves single-cell DNA gel electrophoresis, where DNA damage is quantified based on DNA in the nucleus (comet head) and damaged fragments that travel across the gel (Tail). The extent tail moment (Tail DNA %× Length of Tail) calculated gives insight into the extent of DNA damage. The extent tail moment was analyzed using Open Comet plugin for ImageJ. At least 50 individual cells per treatment were analyzed.

### 1.10 γ-H2AX Immunofluorescence

Phosphorylated Serine (139) on histone variant H2AX were detected using antibodies at a dilution of 1:1000 (Cell signalling, 2577S) as described previously with minor modifications (Sajesh et al., 2015). U251 cells were treated with LNP_SAT1 (80 nM) and after 48 hours were seeded on coverslips (20,000 cells/cm2). After 24 hours, the cells were exposed to 1 Gy radiation and incubated for 6 hours. Cells were fixed with freshly prepared 4% paraformaldehyde (Fisher Scientific) in phosphate buffered saline (PBS; 0.01M; pH 7.4) for 20 minutes. Following fixation, cells were washed with PBS, permeabilized with 0.5% Triton™-X-100 in PBS and incubated with the primary antibody for one hour at room temperature. Cells were washed with 0.1% Triton™-X100 (in PBS) followed by PBS and incubated with the secondary antibody (AlexaFluor™ 488; 1:200; Thermofisher) for one hour at room temperature. Cells were washed with 0.1% Triton™-X100 (in PBS) followed by PBS, and nuclei were counterstained with DAPI (Abcam; ab104139). For microscopy, 30 cells were imaged on an AxioImager™ 2 (Zeiss) equipped with an AxioCam™ HR charge-coupled device (CCD) camera (Zeiss) and a 63× oil immersion plan-apochromat lens (1.4 numerical aperture). Images were acquired with AxioVision™ software and saved as 16-bit Tiff images. Nuclei was imaged on the blue channel (pseudo-colored red for illustration purposes), and γ-H2AX foci were imaged on the green channel (pseudo-colored green for illustration purposes). γ-H2AX foci were enumerated using ImageJ using the plugins as described elsewhere, data were exported into prism for statistical analysis. Images were processed using Imaris™ cell imaging software (Oxford Instruments) to separate channels, and pseudo color nuclei and γ-H2AX. Image panels were generated using Photoshop™ CS5 (Adobe).

### 1.11 BBB-GBM Co-Culture Model

A BBB-GBM co-culture model was used to assess the delivery of the LNP-siSAT1 formulation across brain microvessel endothelial cells, (Norouzi et al., 2020). For these studies, hCMEC/D3 cells (a model of the human blood brain barrier) were grown to confluency on Transwell inserts (0.4 micron pore size), and the inserts were placed in 12-well plates containing U251 tumor cells (approximately 70% confluency). Prior to transfection with LNP-siSAT1, media was removed from the top (donor compartment) and bottom (receiver compartment) of the insert and replaced with transfection media (hCMEC/D3 media described above without FBS and antibiotics). To increase penetration of the LNPs across the hCMEC/D3 monolayers, cadherin peptide (ADTC5; 1 mM) was added to the donor compartment and pre-incubated for 30 minutes at 37° C. After the pre-incubation period, LNP-siSAT1 (40 nM) was added to the donor compartment along with APOE (2 μg/mL) and incubated for two hours at 37° C. with shaking (50 RPM). After two hours, the donor compartment was removed along with the hCMEC/D3 monolayer and Transwell insert, and the U251 cells plated in the receiver compartment were further incubated for 6 hours in the humidified CO₂ incubator to aid transfection. After the 6 hours, the media was changed to complete U251 media and SAT1 knock-down was determined at 48 hours as described above. To assess the integrity of the monolayer, a paracellular permeability marker IR-PEG (0.1 μM) was added to the donor compartment at the start of the transfection treatment. The concentration of IR-PEG in the donor compartment and the receiver compartments were measured fluorometrically (Ex: 485 nm and Em: 528 nm). Permeability was expressed as the percent flux determined by dividing the cumulative concentration of the dye in the receiver compartment (t=2 h) by the concentration in the donor compartment (t=0).

### 1.12 Statistical Analysis

All data are expressed as the mean±standard error of the mean (SEM). Statistical analysis was performed using one-way or two-way ANOVA, followed by Tukey's test. In all studies, p<. 05 was considered statistically significant.

### Example 2: SAT1 Expression in GBM Patients

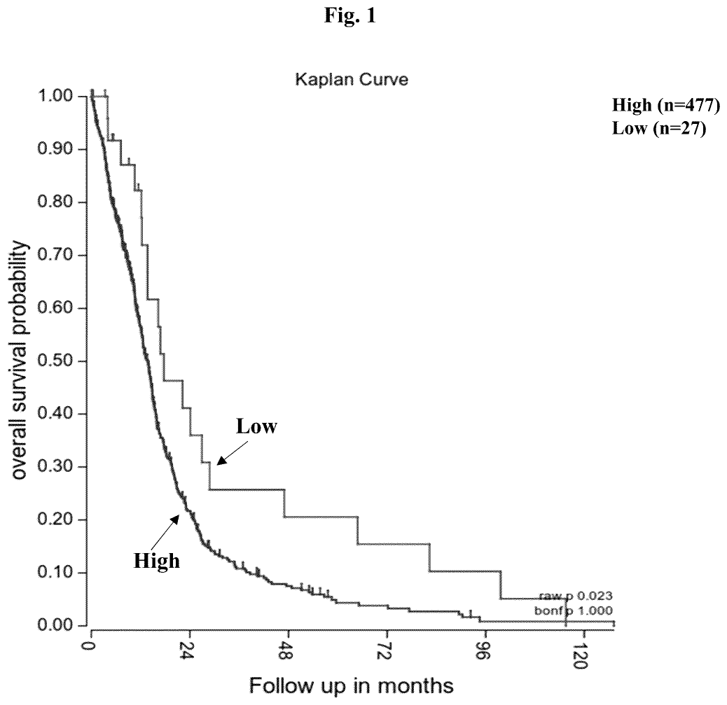

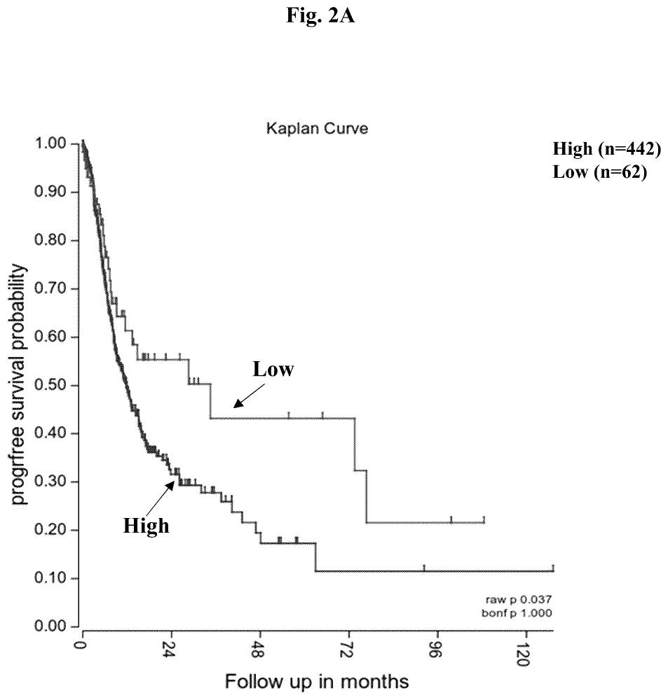

SAT1 gene expression was first assessed in approximately 500 GBM patients and correlation studies were performed for the overall survival probability, as well as progress-free survival probability. GBM patients with a lower expression of SAT1 were found to have a significantly higher probability of overall (FIG. 1) and progress-free survival (FIG. 2). These results strongly indicate that SAT1 may be an important target for GBM treatment, as well as a potential biomarker for GBM diagnosis and/or prognosis.

### Example 3: SAT1 Knockdown Via LNP-siSAT1 Inhibits Viability/Proliferation of U251 GBM Cells

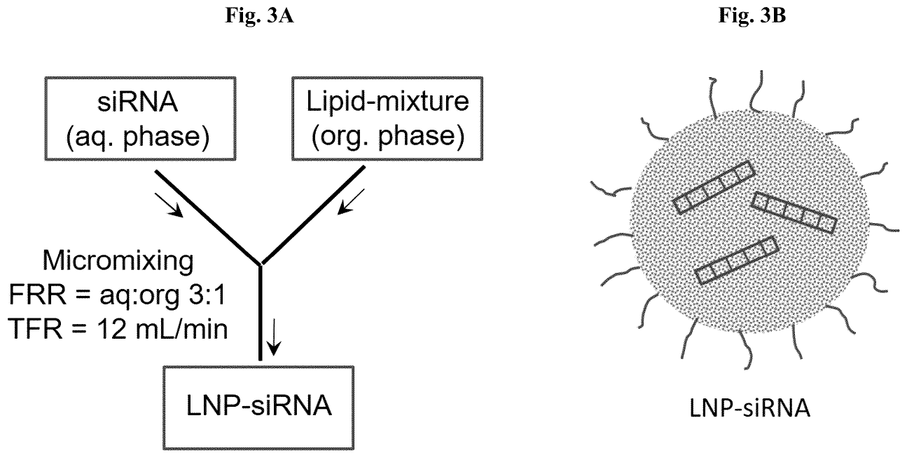

The siSAT1-encapsulated LNP (DODAP/DSPC/cholesterol/DiR/DSPE-PEG) was formulated following the microfluidic mixing procedure outlined in FIGS. 3A and 3B and described in Example 1.3. The hydrodynamic size of the LNP-siSAT1 was estimated to be about 80 nm, with a net neutral surface charge (zeta potential: 0.18±0.42). The particles displayed a polydispersity index (PDI) of 0.16, indicating the monodisperse nature of the nanoparticles. The UV spectroscopic (A260/A280) analysis of siRNA in both LNP-siSAT1 and filtrate showed a high encapsulation efficiency of 100%. The LNP-siRNA formed a highly stable but hazy dispersion in PBS (pH 7.4) and was stored at 4° C. and −80° C. for the short and long term, respectively, without significant loss of stability or biological activity.

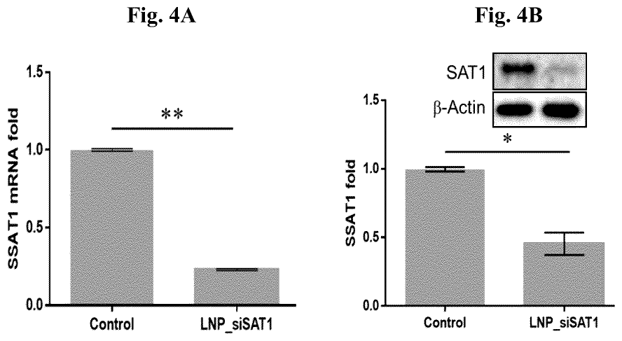

The ability of LNP-siSAT1 to deliver siRNA and knockdown the target SAT1 gene was evaluated in U251 cells. The cells were transfected with LNP-siSAT1 (80 nM siRNA) or a control siRNA (siSCR; scramble siSAT1 sequence) in the presence of APOE (1 μg/mL), which was added to the transfection media to achieve the desired knockdown level of SAT1 knockdown. The SAT1 mRNA and protein levels estimated at 72 hours indicated a knockdown of 78% and 45%, respectively (FIGS. 4A and 4B). SAT1 immunofluorescence studies also confirmed a reduction in SAT1 protein in the knockdown cells compared to cells treated with scrambled siRNA control (data not shown). This level of SAT1 knockdown was achieved for LNP-siSAT1 formulations with an N/P ratio of 15. However, LNP-siSAT1 formulations with N/P ratios of 5 and 10 were also tested but no level of SAT1 mRNA knockdown was observed at these ratios.

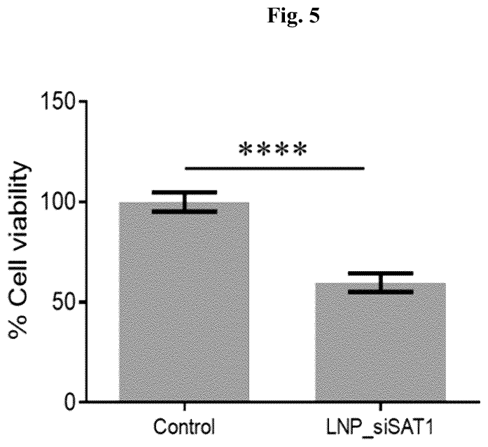

The effects of SAT1 knockdown on U251 cell proliferation/viability was examined by MTT assay as described in Example 1.8. Strikingly, a single transfection event with our LNP-siSAT1 formulation resulted in a 40% reduction in viable U251 cells that was observable even six days post-transfection, in comparison to cells transfected with the negative control LNP-siSCR (“control”; FIG. 5). Such an inhibitory effect on a glioblastoma cell growth was not observed in previous studies attempting to transiently knockdown SAT1 mRNA expression using other siRNA delivery formulations (Brett-Morris et al., 2014).

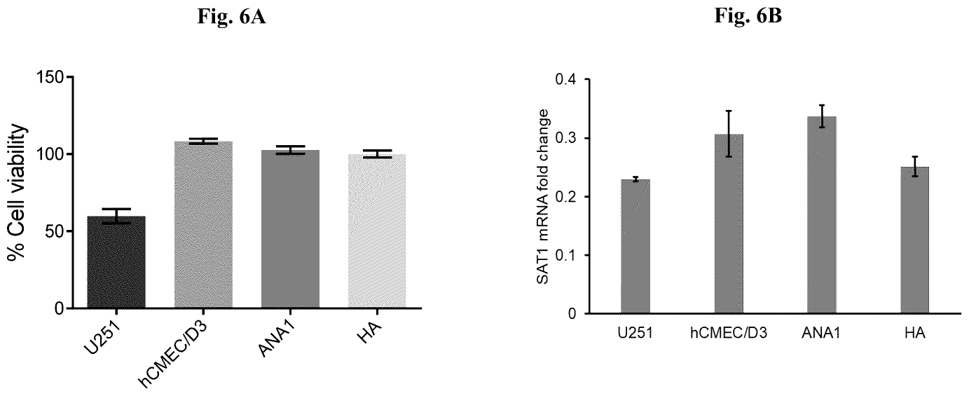

We further investigated the impact of our LNP-siSAT1 formulation on other non-glioblastoma cell lines that are relevant in the context of developing a brain therapeutic: brain microvascular endothelial cells (hCMEC/D3), primary human astrocytes (HA), and macrophage cells (ANA-1). Interestingly, while a similar magnitude of SAT1 mRNA knockdown was achieved in hCMEC/D3, HA and ANA-1 cells (FIG. 6B), the reduction of SAT1 expression did not affect the viability/proliferation of these non-glioblastoma cells (FIG. 6A). These results suggest a degree of specificity of LNP-siSAT1-mediated SAT1 knockdown on the viability/proliferation of U251 glioblastoma cells.

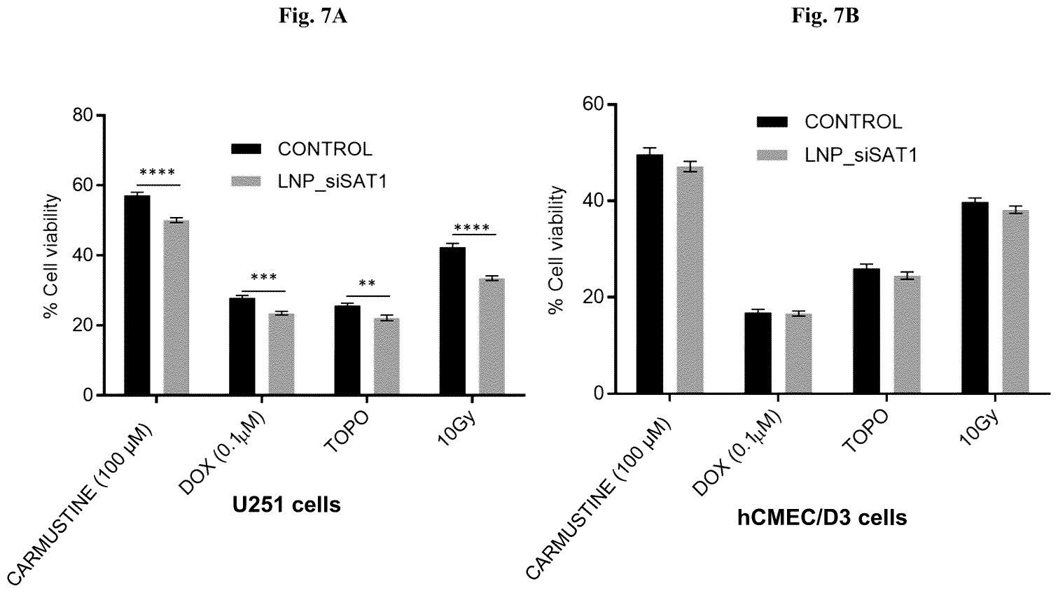

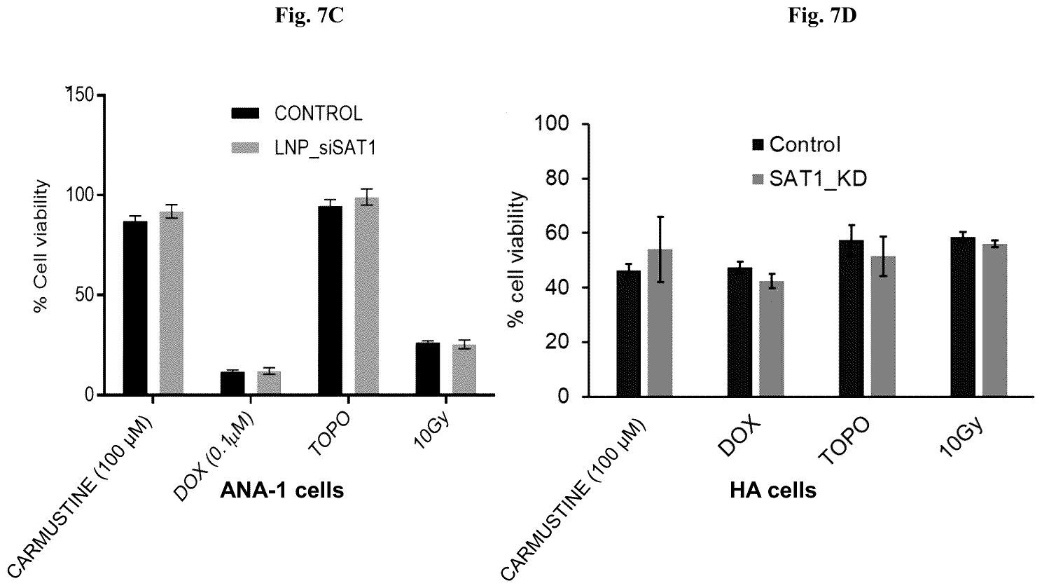

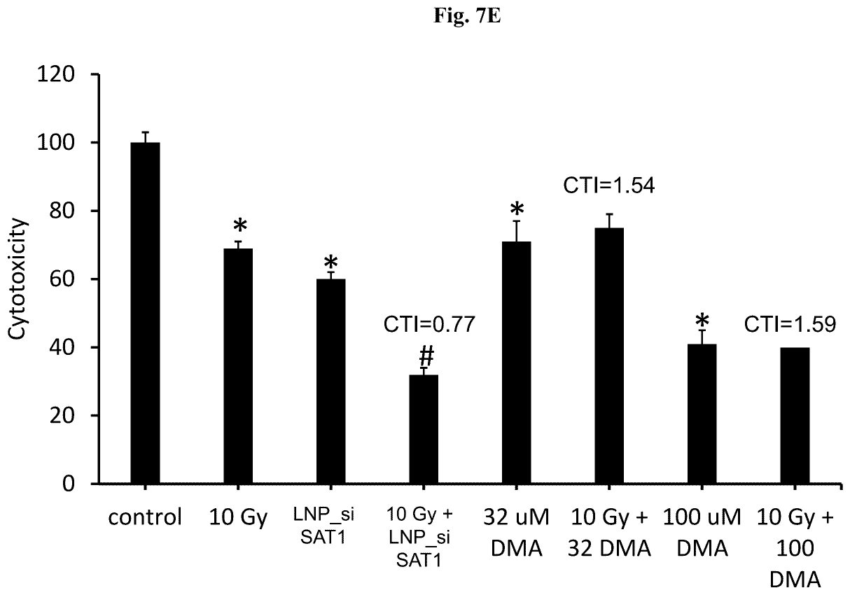

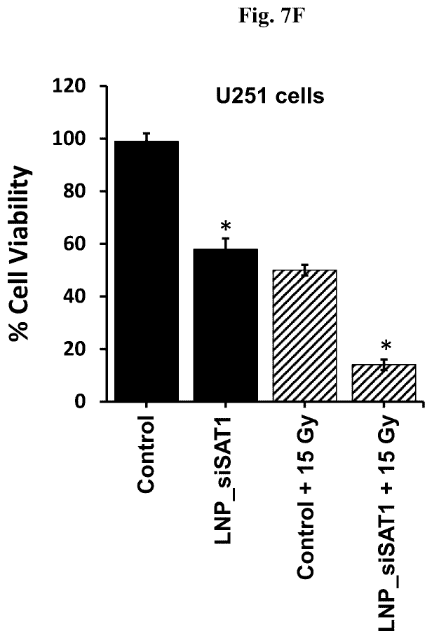

Next, we assessed the effects of LNP-siSAT1 on cell viability/proliferation following radiation or chemotherapeutic exposure in the four different cell types (i.e., U251, hCMEC/D3, HA, and ANA-1) via MTT assay (FIG. 7A-7D). For radiation treatment (“10 Gy”), U251 cells that were transfected with the negative control LNP-siSCR formulation showed 52% cell viability compared to 35% cell viability in cells treated with LNP-siSAT1 formulation (FIG. 7A). Similar effects were observed following treatment with chemotherapeutic agents known to cause DNA double-strand breaks was (doxorubicin (DOX), carmustine, and topotecan (TOPO); FIG. 7A-7D). Of note, the lowest cell viabilities/proliferation observed in U251 cells were with SAT1 knockdown combined with DOX or TOPO treatment (FIG. 7A). Furthermore, ANA-1 macrophage cells were particularly sensitive to DOX treatment, suggesting that other chemo treatments (e.g., Carmustine or TOPO) may be considered if the negative impact on macrophage cells is to be reduced.

A further experiment in U251 cells (40,000 cells seeded/well in 24-well plate; 80 nM LNP-siSCR (“Control) or LNP-siSAT1) was performed using a higher dose of radiation (15 Gy at 28 h and 15 Gy at 72 h). The results in FIG. 7F show an even greater effect on the inhibition of U251 cell viability/proliferation following SAT1 knockdown alone (“LNP_siSAT1”) or combined with radiation (“LNP_siSAT1+15 Gy”), as compared to radiation treatment alone (“Control+15 Gy”). The synergistic effect of SAT1 knock-down combined with radiation was also observed in other brain cancer cell lines (including 42-MG-BA cells and LN229), although the inhibition of cell viability/proliferation following SAT1 knockdown alone was less evident in these slower growing cells, suggesting that the effect seen with U251 cells is due to the inhibition of cell proliferation.

### Example 4: LNP-siSAT-Mediated Knockdown of SAT1 Decreases DNA Damage Repair in U251 GBM Cells

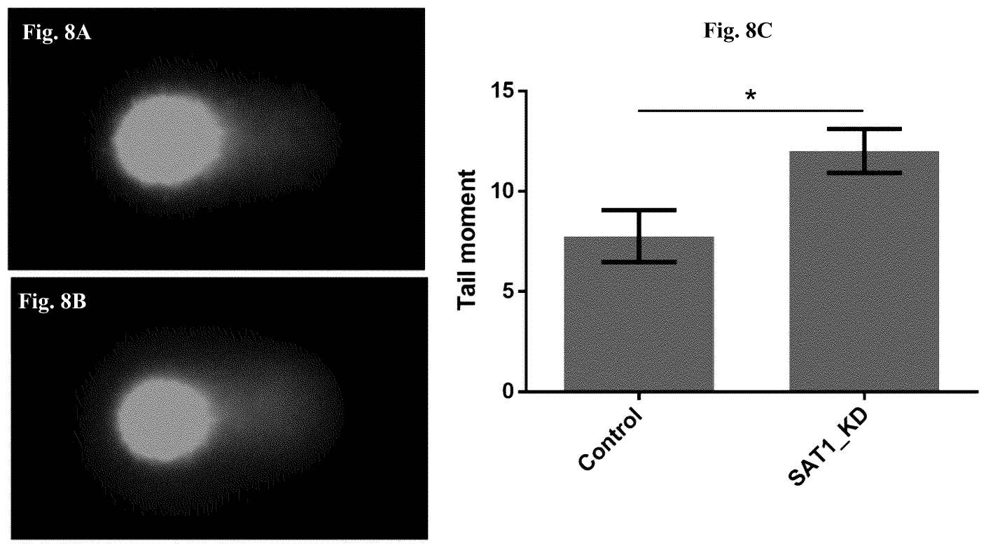

The impact on DNA repair was examined using a comet assay to explore the cellular mechanisms responsible for the effects of LNP-siSAT-mediated SAT1 knockdown in the U251 GBM cell line (FIG. 8A-8C). Here, the negative control LNP-siSCR-(FIG. 8A) and LNP-siSAT-treated (FIG. 8B) cells were exposed to 10 Gy radiation and the Comet assay was performed after six hours. The calculated extent tail moments in the SAT1 knockdown cells were found to be 1.5-fold higher than those of the control cells, indicating a significant reduction in DNA damage repair in the LNP-siSAT-treated cells (FIG. 8C).

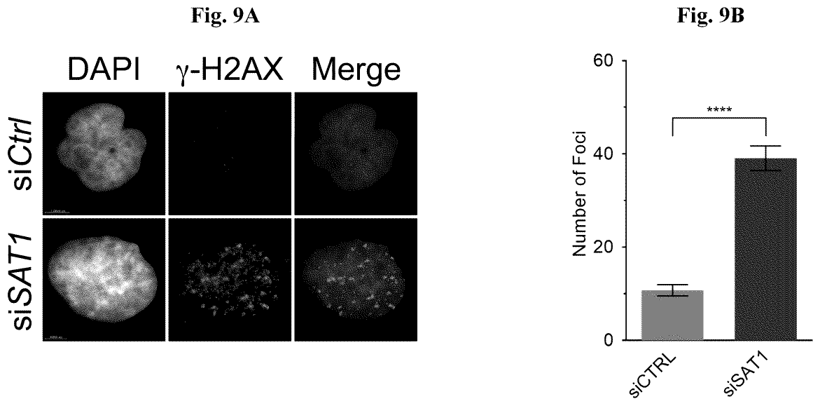

γ-H2AX is a well-established marker for DNA double-strand breaks and repair. At the onset of DNA double strand break (DSB), an early cellular response is the rapid phosphorylation of H2AX at Ser139 to form γ-H2AX, followed by the recruitment of DNA damage repair enzymes. We evaluated the recruitment of DNA damage repair machinery via the appearance of γ-H2AX-positive foci by immunofluorescence microscopy (FIG. 9A). In SAT knockdown U251 cells exposed to 1 Gy radiation, we found a 4-fold higher number of γ-H2AX foci after six hours compared to the negative control-treated cells, which is normally indicative of impaired DNA repair capacity (FIG. 9B). Together, these findings suggest that higher SAT1 expression is correlated with improved DNA repair in U251 cells, which may confer some resistance to radiation and chemotherapy.

### Example 5: LNP-Mediated Delivery of siSAT1 Across a Model of the Blood-Brain Barrier

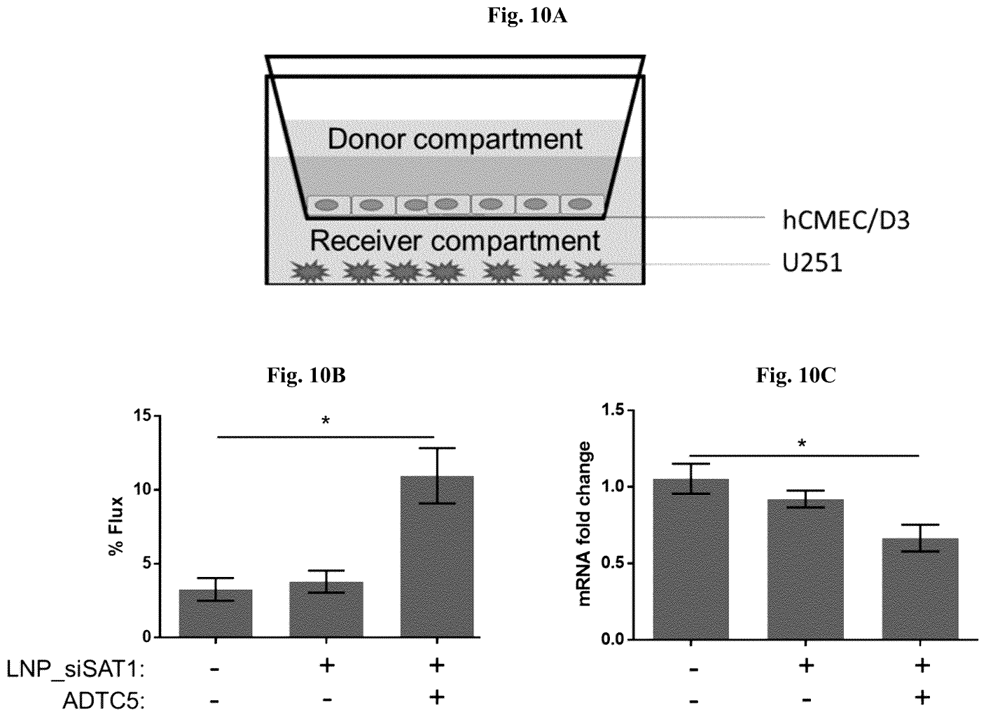

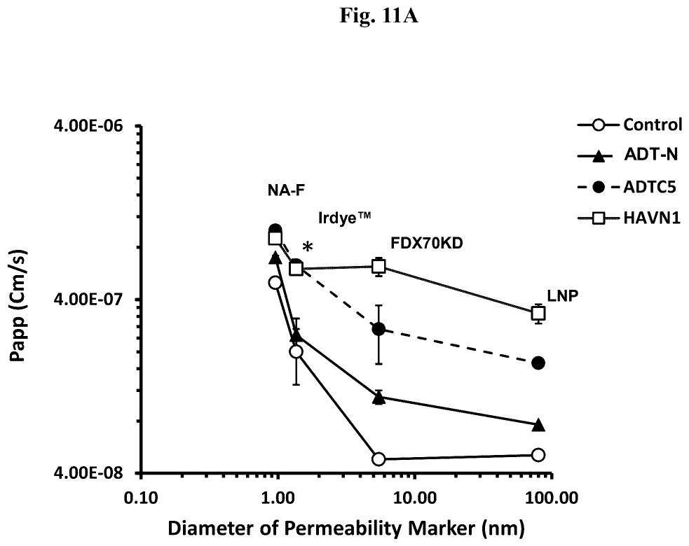

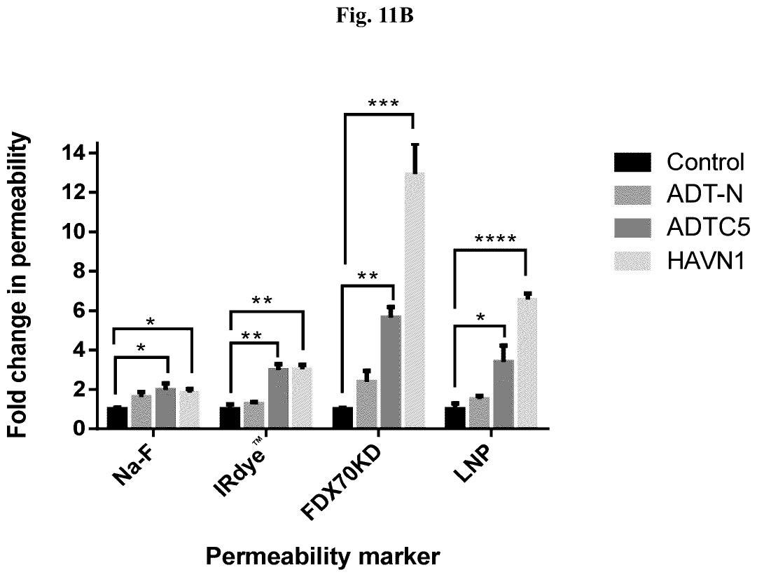

Monolayers formed by hCMEC/D3 cells are widely used as a model for the blood-brain barrier (BBB). We evaluated the ability of LNP-siSAT1 to deliver siRNA across a monolayer of hCMEC/D3 cells and into U251 cells using the BBB-GBM co-culture model as described in Example 1.11, and as shown schematically in FIG. 10A. The integrity of the hCMEC/D3 monolayer was tracked based on the percent flux of a 35-kDa IRdye-PEG permeability marker (FIG. 10B), and the ability of LNP-siSAT1 to cross the monolater and knock-down of SAT1 was assessed at the mRNA level (FIG. 10C).

Cadherin peptides have been shown to permit delivery of small molecules and even some recombinant proteins (e.g., immunoglobulins) across the BBB (Ulapane et al., 2019). Here, we explored the ability of the cadherin peptide, ATDC5, to mediate delivery of LNP-siSAT1 particles. As shown in FIG. 10B, ATDC5-treated hCMEC/D3 monolayers displayed a 2.8-fold higher flux of IRdye-PEG compared to the control cells. The higher flux of the macromolecule permeability marker indicated that the ADTC5 disruption was successful, thereby enabling the paracellular diffusion of the hydrophilic dye. The permeability of LNP-siSAT1 across the hCMEC/D3 monolayers was evaluated based on the fold decrease in SAT1 mRNA in co-cultured U251 cells. In the group with ADTC5 disruption, a knockdown of SAT1 mRNA of ˜37% was observed (FIG. 10C). A small but detectable reduction in SAT1 mRNA expression was also seen for cells treated with LNP-siSAT1 without the ADTC5 treatment (FIG. 10C). These results suggest that that the LNP-siSAT1 formulation, particularly when combined with agents that include transient BBB permeability, may be suitable for delivering LNP-encapsulated siRNA to GBM targets in the brain.

### Example 6: Effect of Cadherin Peptides on BBB Permeability of Different Sized Particles

Next, different cadherin peptides were tested for their effect on BBB permeability of different sized particles for improving the delivery LNP-encapsulated siSAT1 in the brain. Various cadherin peptides have been previously synthesized, including ones derived from the bulge regions (HAV peptides) or groove regions (ADT peptides) of the E-cadherin extracellular 1 domain (EC-1 domain) (e.g., Ulapane et al., 2019; Sinaga et al., 2002; WO2020257745A1). Combinations of these peptides, such as ADTHAV peptides, have also been previously synthesized. In the present Example, cadherin peptides derived from different regions of the EC1 domain of E-cadherin were assessed for their ability to permeabilize the BBB and deliver different sized particles. The cadherin peptides tested herein include: ADTC5, which is derived from the C-terminal region of the binding domain of the extracellular-1 (EC-1) domain of E-cadherin; ADT-N, which is derived the N-terminal region of the binding domain of the EC-1 domain, and HAVN1, which is derived from the bulge region of the EC-1 domain having components derived from both N- and C-terminal regions of the binding domain.

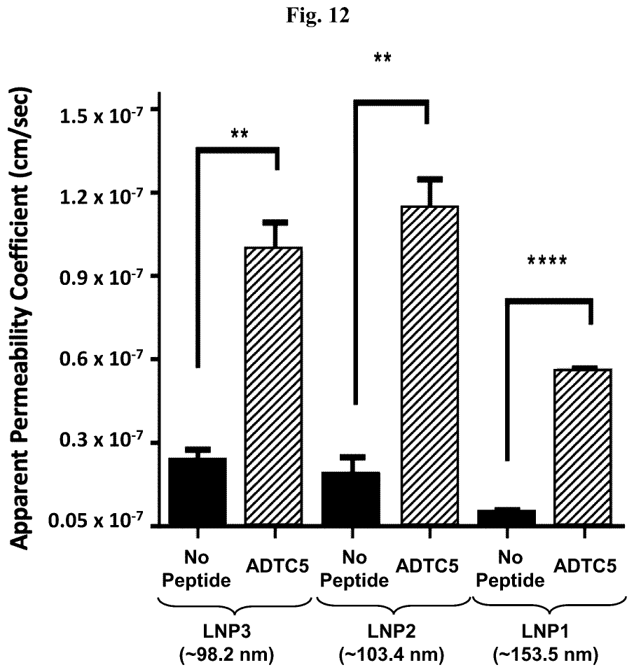

As shown in FIGS. 11A and 11B, both ADTC5 and HAVN1 enhanced the delivery of the four differently sized markers, Na-F (˜0.95 nm), Irdye™ 800 CW (˜1.4 nm), fluorescein labeled dextran (FDX70KD, ˜5.5 nm), and the lipid nanoparticles as described in Examples 1-5 (LNP, ˜80 nm), across the BBB in the model with respect to control (no cadherin peptide). The peptides ADTC5 and HAVN1 enabled significantly higher relative permeability of the large ˜80-nm LNPs over the control (FIG. 11B).

Next, different sized lipid nanoparticles containing siSAT1 (LNP1-3) were produced to confirm the enhancement of delivery across the BBB by the different cadherin peptides. The characteristics of the LNPs used in this experiment are shown in Table 2, as determined by dynamic light scattering and zeta potential analysis. LNP1-3 were produced using the same methods as described in Example 1, and were shown to have a neutral in surface charge and had an optimal polydispersity index (did not aggregate).

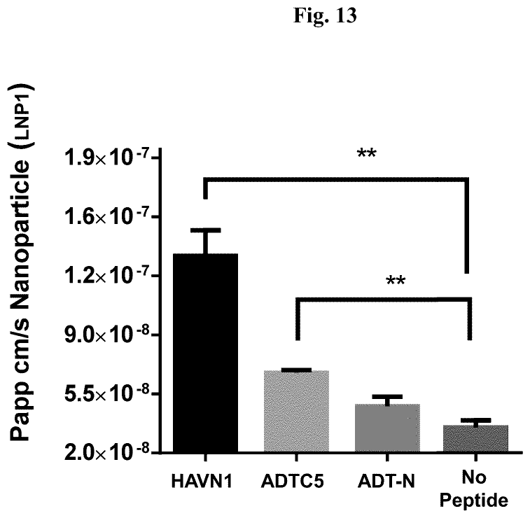

TABLE 2
Characteristics of LNPs used in FIGS. 12 and 13

| | Total Flow | Size | | Surface |
|---|---|---|---|---|
| LNP | Rate (TFR) | (nm) | PDI | Charge |
| LNP1 | 4 | 153.5 | 0.208 | Neutral |
| LNP2 | 8 | 103.4 | 0.270 | Neutral |
| LNP3 | 12 | 98.2 | 0.286 | Neutral |

ADTC5 was shown to enhance delivery of each different sized LNP1-3 across the BBB (FIG. 12). Furthermore, delivery of LNP1 (the largest of the LNPs) across the BBB was significantly enhanced by HAVN1 and ADTC5 (FIG. 13).

These results highlight the potential limitations of different cadherin peptides with respect to the size and/or nature of the cargoes to be delivered, and suggest that cadherin peptides such as ADTC5 and HAVN1, which include a region derived from the C-terminal region of the binding domain of the EC-1 domain of E-cadherin, may be particularly suitable for the delivery of LNP cargoes across the BBB. Furthermore, these results suggest that cadherin peptides, such as ADTC5 and HAVN1, enable delivery of LNPs up to or greater than 150 nm in diameter. These results are unexpected, given that it has been previously reported that there is a cut-off size of molecules that can be delivered by ADTC5 across the BBB, with 220 kDa fibronectin being reported to be undeliverable (Ulapane et al., 2019a and Ulapane et al., 2019b).

### Example 7: SAT1 Activity and/or Expression as a Potential Biomarker for GBM

Next, we evaluated the potential for using SAT1 activity and/or expression as biomarker for GBM diagnosis, detection, and/or prognosis. As discussed in Example 2, SAT1 expression is enhanced in patients with GBM, and elevated SAT1 expression is associated with a lower overall survival and progress-free survival probability and time in GBM. Therefore, assessing SAT1 expression and likely activity may be a potential indicator of the presence and/or severity of GBM in patients.

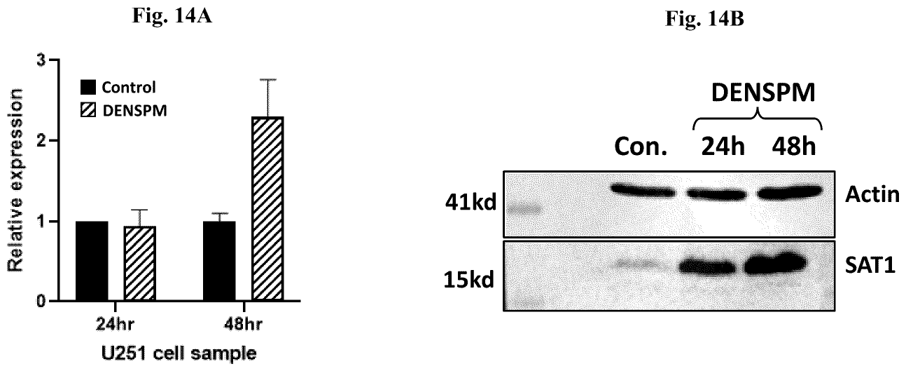

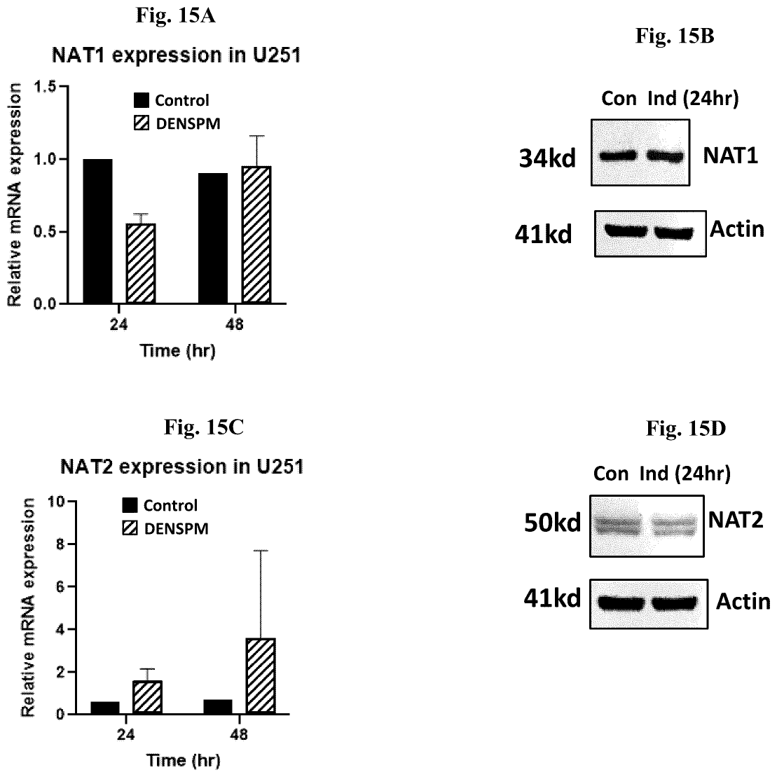

SAT1 expression was next evaluated in U251 cells. Baseline levels of SAT1 gene and protein expression were observed in untreated U251 cells (“control”), and were enhanced in the presence of the SAT1 small molecule activator, N(1),N(11)-diethylnorspermine (DENSPM) after 24 and 48 hours (FIGS. 14A and 14B). Conversely, N-acetyltransferases NAT1 and NAT2 gene and protein expression levels in U251 cells did not significantly change either at baseline or after treatment with DENSPM (FIGS. 15A-15D).

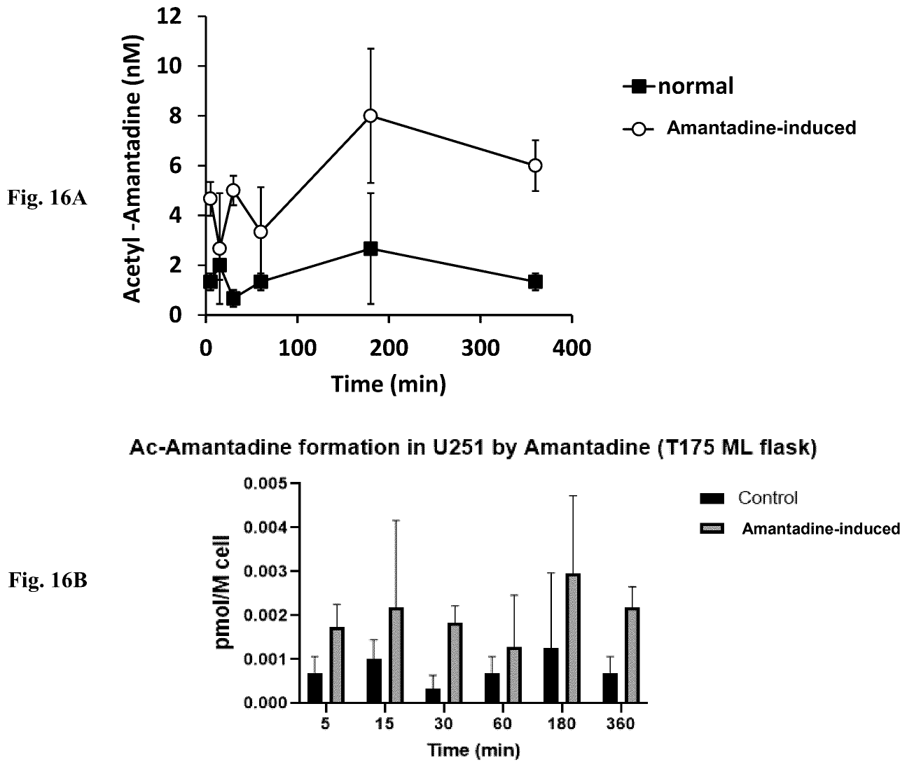

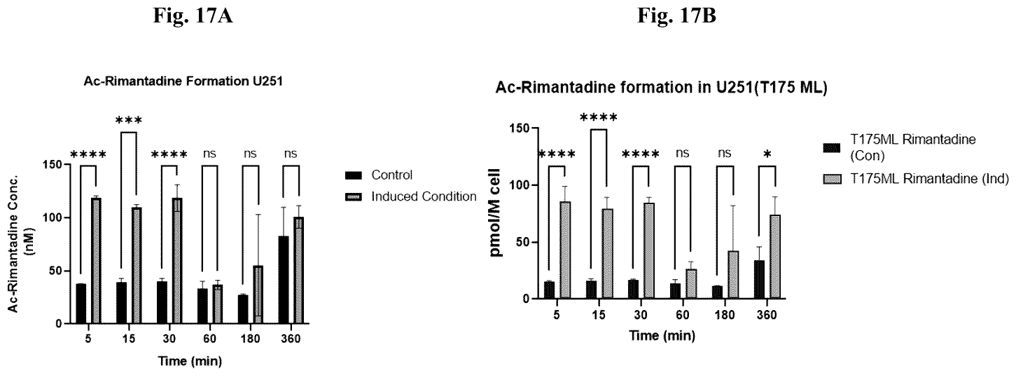

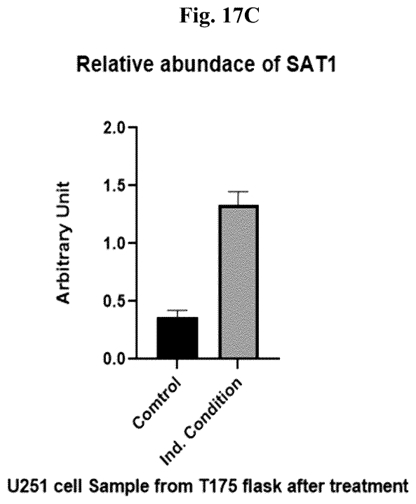

Next, SAT1 activity was assessed in U251 cells via production of acetylated-amantadine after treatment with the SAT1 substrate amantadine, or the production of acetylated-rimantadine after treatment with the SAT1 substrate rimantadine. Baseline Ac-amantadine and Ac-rimantadine levels were observed in untreated U251 cells and were increased in the presence of their respective substrates (FIGS. 16A-16B and 17A-17C). Ac-amantadine and Ac-rimantadine levels were detected as early as 5 minutes after induction and in some cases as early as 3 minutes (data not shown).

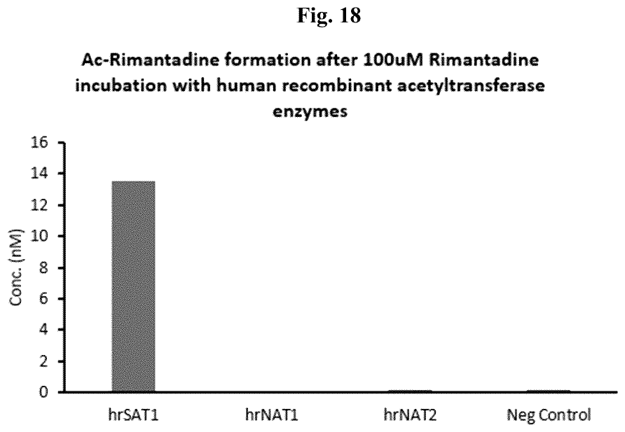

Next, human recombinant SAT1, NAT1, or NAT2 enzymes were incubated with rimantadine, in solution, and the Ac-rimantadine metabolite levels were measured. As shown in FIG. 18, only hrSAT1 metabolized rimantadine into Ac-rimantadine.

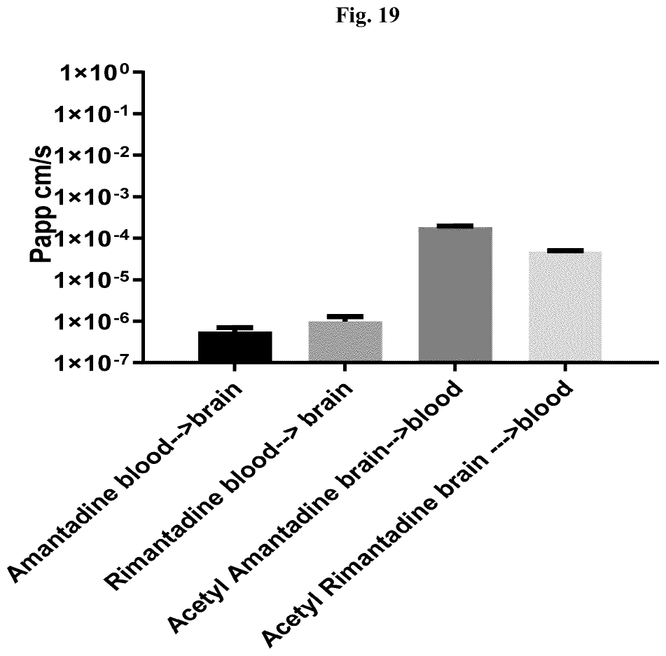

Finally, the bidirectional BBB permeability of amantadine and rimantadine, as well as their metabolites, was evaluated in the previously described model. As shown in FIG. 19, both amantadine and rimantadine were able to cross the BBB from blood to the brain. Rimantadine was shown to be slightly more permeable. Furthermore, both Ac-amantadine and Ac-rimantadine were able to cross the BBB from the brain to blood efficiently.

These data demonstrate that SAT1 expression and activity can be used as a specific and effective biomarker for GBM detection, diagnosis, and/or prognosis. Furthermore, to better detect SAT1 expression and activity and confirm the diagnosis or prognosis in potential GBM patients, both amantadine or rimantadine can be administered and their respective metabolites can be subsequently measured (e.g., in the blood).

### Example 8: Summary and Discussion

The lipid composition of the LNPs plays a significant role in determining the drug entrapment efficiency, size, surface charge and blood circulation half-life. A mixture of lipids was used to achieve the desired physicochemical and drug loading/delivery properties. For our formulation, we focused on DODAP, DSPC, cholesterol, and DSPE-PEG2000 as the main lipid components. The LNP-siSAT1 formulation described herein displayed a high siRNA encapsulation efficiency, low polydispersion index, and neutral surface charge. Initially, cationic DOTAP-based LNPs were formulated and tested but even negative control formulations were found to be cytotoxic. For example, an N/P ratio of 10 was considerably toxic to U251 cells, while an N/P ratio of 5 killed around 30% of U251 cells. In contrast, control ionizable DODAP-based LNPs used herein exhibited minimal cytotoxicity on U251 (FIG. 5) and other cells tested (FIG. 6A) in the absence of SAT1 knockdown.

The LNP-siSAT1 formulation described herein effectively delivered siSAT1 in a GBM cell line producing significant knockdown of SAT1 at both the mRNA and protein levels. Strikingly, reduced SAT1 protein levels negatively affected of U251 glioblastoma cell growth/viability, which was not observed in previous studies attempting to transiently knockdown SAT1 mRNA expression using other siRNA delivery formulations (Brett-Morris et al., 2014). A degree of sensitization towards radiation and chemotherapy was also observed upon SAT1 knockdown with the LNP-siSAT1 formulation described herein, although the magnitude of sensitization was lower than that previously reported by others using other siRNA delivery formulations (Brett-Morris et al., 2014). Interestingly, SAT1 knockdown using the LNP-siSAT1 formulation described herein did not negatively impact the viability/proliferation of brain microvascular endothelial, astrocyte and macrophage cell lines. These findings point favorably to the use of the LNP-siSAT1 formulations for the development therapeutics to be delivered to the brain. The results from the BBB-GBM co-culture model show that LNP-siSAT1 particles are suitable for delivery across the BBB into GBM cells efficiently and at therapeutic concentrations, particularly when combined with transient modulation of BBB permeability (e.g., using cadherin binding peptide).

### REFERENCES

Brett-Morris et al., “The Polyamine Catabolic Enzyme SAT1 Modulates Tumorigenesis and Radiation Response in GBM.” Cancer Research 2014, 74 (23), 6925-6934.
Kulkarni et al., “On the Formation and Morphology of Lipid Nanoparticles Containing Ionizable Cationic Lipids and siRNA.” ACS Nano 2018, 12 (5), 4787-4795 (Kulkarni et al., 2018A).
Kulkarni et al., “Lipid Nanoparticles Enabling Gene Therapies: From Concepts to Clinical Utility.” Review Nucleic Acid Ther. 2018 June; 28 (3): 146-157 (Kulkarni et al., 2018B).
Norouzi et al., “Doxorubicin-loaded iron oxide nanoparticles for glioblastoma therapy: a combinational approach for enhanced delivery of nanoparticles.” Scientific reports 2020, 10 (1), 11292.
On et al., “Modulation of Blood-Brain Barrier Permeability in Mice Using Synthetic E-Cadherin Peptide.” Molecular Pharmaceutics 2014, 11 (3), 974-981.
Sajesh et al., “Targeting SOD1 induces synthetic lethal killing in BLM- and CHEK2-deficient colorectal cancer cells.” Oncotarget 2015, 6 (29), 27907-27922.
Sinaga et al. “Increasing Paracellular Porosity by E-Cadherin Peptides: Discovery of Bulge and Groove Regions in the EC1-Domain of E-Cadherin.” Pharmaceutical Research, Vol. 19, No. 8, August 2002.
Schmittgen et al., “Analyzing real-time PCR data by the comparative C-T method.” Nature Protocols 2008, 3 (6), 1101-1108.
Ulapane et al., “Improving In Vivo Brain Delivery of Monoclonal Antibody Using Novel Cyclic Peptides”. Pharmaceutics 2019, 11, 568 (Ulapane et al., 2019a).
Ulapane et al., “In Vivo Brain Delivery and Brain Deposition of Proteins with Various Sizes”. Pharmaceutics 2019, 11, 568 (Ulapane et al., 2019b).

1. A biocompatible lipid nanoparticle composition comprising, or consisting essentially of, an siRNA encapsulated in a lipid component, the lipid component comprising a mixture of:
(a) an ionizable cationic lipid (e.g., ionizable cationic unsaturated lipid) having a polar head group with a pKa of below 7;
(b) a PEGylated lipid;
(c) a sterol; and
(d) a phospholipid.

2. The biocompatible lipid nanoparticle composition of claim 1, wherein the nanoparticles have:
(i) a hydrodynamic size of about 60 to about 160 nm, about 60 to about 155 nm, about 60 to about 150 nm, about 65 to about 95 nm, about 70 to about 90 nm, about 75 to about 85 nm, or about 80 nm;
(ii) a net neutral surface charge at physiological pH (e.g., zeta potential of below 0.6, 0.5, 0.4, 0.3, or 0.2);
(iii) a polydispersity index (PDI) of below 0.2;
(iv) an N/P ratio of between 12 to 20, 13 to 19, 13 to 18, 13 to 17, 14 to 16, or about 12, 13, 14, 15, 16, 17, 18, 19, or 20; or
(v) any combination of (i) to (iv).

3. The biocompatible lipid nanoparticle composition of claim 1, wherein:
(a) the ionizable cationic unsaturated lipid is: 1,2-dioleoyl-3-dimethylammonium-propane (DODAP); 1,2-dioleyloxy-N,N-dimethyl-3-aminopropane (DODMA); heptatriaconta-6,9,28,31-tetraen-19-yl 4-(dimethylamino) butanoate (DLin-MC3-DMA or MC3); 2,2-dilinoleyl-4-(2-dimethylaminoethyl)-[1,3]-dioxolane (DLin-KC2-DMA or KC2); or other pharmaceutically acceptable ionizable cationic unsaturated lipid; or any combination thereof;
(b) the PEGylated lipid is (1,2-distearoyl-sn-glycero-3-phosphorylethanolamine)-PEG (DSPE-PEG); (1,2-dimyristoyl-rac-glycero-3-methoxy)-PEG (DMG-PEG); or other pharmaceutically suitable PEGylated lipid;
(c) the sterol is cholesterol or other pharmaceutically suitable sterol;
(d) the phospholipid is distearoylphosphatidylcholine (DSPC); 1,2-dioleoyl-sn-glycero-3-phosphocholine (DOPC); 1-palmitoyl-2-oleoyl-glycero-3-phosphocholine (POPC); or other pharmaceutically suitable phospholipid; or
(e) any combination of (a) to (d).

4. The biocompatible lipid nanoparticle composition of claim 3, wherein the lipid component comprises a molar ratio of between 30 to 70, 35 to 65, 40 to 60, 47 to 57, 45 to 55%, or about 35, 40, 45, 50, 55, 60, 65, or 70% of the ionizable cationic unsaturated lipid.

5. The biocompatible lipid nanoparticle composition of claim 1, wherein the siRNA comprises an siRNA for inhibiting expression of Spermidine/spermine N1-acetyltransferase 1 (SAT1).

6. The biocompatible lipid nanoparticle composition of claim 1, which is prepared by microfluidic mixing of suitable volumes of an aqueous phase and an organic phase, the aqueous phase comprising the siRNA dissolved in an acidic buffer (e.g., acetate buffer), and the organic phase comprising the lipid component ingredients dissolved in ethanol, followed by dilution in a buffer at physiologic pH.

7-8. (canceled)

9. A method for inhibiting the growth of brain tumor cells, the method comprising contacting the brain tumor cells with the biocompatible lipid nanoparticle composition as defined in claim 1 comprising an siRNA for inhibiting expression of Spermidine/spermine N1-acetyltransferase 1 (SAT1), encapsulated in a lipid component.

10. The method of claim 9, wherein the brain tumor cells are grade 1, 2, 3 and/or 4 brain tumors, gliomas, astrocytomas, glioblastoma cells (e.g., glioblastoma cells characterized by overexpression of SAT1; highly proliferative glioblastoma cells; glioblastoma cells deficient in DNA damage repair mechanisms; glioblastoma cells resistant to temozolomide (TMZ)).

11. The method of claim 9, wherein the lipid component comprises a mixture of:
(a) an ionizable cationic lipid (e.g., ionizable cationic unsaturated lipid) having a polar head group with a pKa of below 7;
(b) a PEGylated lipid;
(c) a sterol; and
(d) a phospholipid.

12. (canceled)

13. The method of claim 9, where the brain tumor cells are in a subject to be treated and the contacting is performed by administering the lipid nanoparticle composition in the brain of the subject, thereby bypassing the blood-brain barrier.

14. The method of claim 9, where the brain tumor cells are in a subject to be treated and the lipid nanoparticle composition is administered intravenously in combination with a blood-brain barrier permeabilizing agent.

15. The method of claim 14, wherein the blood-brain barrier permeabilizing agent is a cadherin binding peptide (e.g., a linear or cyclic ADTC5, HAVN1, HAVN2, ADTHAV, HAV6, HAV4, CHAVc1, or cHAVc3 peptide).

16. The method of claim 9, wherein the lipid nanoparticle composition is administered in combination with a chemotherapy and/or radiation.

17. The method of claim 16, wherein the chemotherapy comprises an alkylating agent (e.g., carmustine, temozolomide), a topoisomerase inhibitor (e.g., topotecan), an anthracycline (e.g., doxorubicin), or any combination thereof.

18. The method of claim 16, wherein the chemotherapy lacks an anthracycline (e.g., doxorubicin).

19-21. (canceled)

22. A method for increasing the delivery of an RNA payload across the blood-brain barrier of a subject, the method comprising: providing a biocompatible lipid nanoparticle composition comprising the RNA payload encapsulated therein; administering the lipid nanoparticle composition intravenously to the subject in combination with a cadherin binding peptide that transiently increases blood-brain barrier permeability.

23. The method of claim 22, wherein the biocompatible lipid nanoparticle composition comprises a mixture of:
(a) an ionizable cationic lipid (e.g., ionizable cationic unsaturated lipid) having a polar head group with a pKa of below 7;
(b) a PEGylated lipid;
(c) a sterol; and
(d) a phospholipid.

24-32. (canceled)

33. A method for producing or modifying a glioblastoma test or a test for detecting glioblastoma, the method comprising adding or integrating into said test quantifying a panel of metabolites in a biological sample from a subject having or suspected of having glioblastoma, the panel comprising one or more corresponding metabolites of substrates of Spermidine/spermine N1-acetyltransferase 1 (SAT1).

34-42. (canceled)

43. The method of claim 9, wherein the nanoparticles have:
(i) a hydrodynamic size of about 60 to about 160 nm, about 60 to about 155 nm, about 60 to about 150 nm, about 65 to about 95 nm, about 70 to about 90 nm, about 75 to about 85 nm, or about 80 nm;
(ii) a net neutral surface charge at physiological pH (e.g., zeta potential of below 0.6, 0.5, 0.4, 0.3, or 0.2);
(iii) a polydispersity index (PDI) of below 0.2;
(iv) an N/P ratio of between 12 to 20, 13 to 19, 13 to 18, 13 to 17, 14 to 16, or about 12, 13, 14, 15, 16, 17, 18, 19, or 20; or
(v) any combination of (i) to (iv).

44. The method of claim 22, wherein the cadherin binding peptide is or comprises linear or cyclic ADTC5, HAVN1, HAVN2, ADTHAV, HAV6, HAV4, CHAVc1, or cHAVc3.


SEQUENCE LISTING (6 sequences)

SEQ ID NO: 1 - RNA, 21 nt, synthetic construct
  source 1..21: mol_type = other RNA
  modified_base 20..21: mod_base = OTHER; note = thymine
ccatccatcaacttctatatt

SEQ ID NO: 2 - RNA, 21 nt, synthetic construct
  source 1..21: mol_type = other RNA
  modified_base 20..21: mod_base = OTHER; note = thymine
tatagaagttgatggatggtt

SEQ ID NO: 3 - DNA, 20 bp, synthetic construct
  source 1..20: mol_type = other DNA
ctccggaaggacacagcatt

SEQ ID NO: 4 - DNA, 21 bp, synthetic construct
  source 1..21: mol_type = other DNA
acctcattgcaacctggctta

SEQ ID NO: 5 - DNA, 20 bp, synthetic construct
  source 1..20: mol_type = other DNA
aaacggctaccacatccaag

SEQ ID NO: 6 - DNA, 20 bp, synthetic construct
  source 1..20: mol_type = other DNA
cctccaatggatcctcgtta